(19) United States
(12) Patent Application Publication
(10) Pub. No.: US 2023/0241158 A1
(43) Pub. Date: Aug. 3, 2023

(54) Hydrophilic Degradable Microspheres for Local Delivering of Glycopeptide Antibiotics and Polycationic Peptide Antibiotics

(71) Applicants: Occlugel, Les Ulis (FR); Centre National de la Recherche Scientifique (CNRS), Paris (FR); Université Paris-Saclay, Gif Sur Yvette (FR)

(72) Inventors: Anne Beilvert, Massy (FR); Emel Corus, Janvry (FR); Laurent Bedouet, Les Mans (FR); Laurence Moine, Saint Cloud (FR)

(21) Appl. No.: 18/003,174

(22) PCT Filed: Jul. 7, 2021
(86) PCT No.: PCT/EP2021/068910

§ 371 Date: Dec. 23, 2022

(30) Foreign Application Priority Data
Jul. 7, 2020 (EP) 20305778.1

(51) Int. Cl. A61K 38/14; A61K 9/16; A61K 38/12; A61K 38/17

(52) U.S. Cl. CPC A61K 38/14; A61K 9/1635; A61K 38/12; A61K 38/1709

(57) ABSTRACT

The invention relates to a composition comprising an effective amount of a peptides antibiotic at least one hydrophilic degradable microsphere comprising a crosslinked matrix, and a pharmaceutically acceptable carrier for administration by injection, the crosslinked matrix being based on at least a) between 10 mol % and 90 mol % of hydrophilic monomer of general formula (I); b) between 0.1 and 30 mol % of a cyclic monomer of formula (II); and c) between 5 mol % and 90 mol % of one degradable block copolymer cross-linker, wherein the degradable block copolymer crosslinker is linear or star-shaped and presents (CH₂═(CR₁₁))-groups at all its extremities and wherein the degradable block copolymer crosslinker has a partition coefficient P of between −3 and 11.20. The invention also relates to such a composition for use for preventing and/or treating infectious diseases, in particular mammal infectious diseases, by local delivery.

### FIELD OF THE INVENTION

The present invention relates to hydrophilic degradable microspheres for local delivering of a peptide antibiotic. In particular, the present invention relates to compositions comprising an effective amount of a peptide antibiotic and hydrophilic degradable microspheres. The present invention also relates to said compositions for use for preventing and/or treating infectious diseases, in particular mammal infectious diseases, by local delivery.

### TECHNICAL BACKGROUND

To treat bacterial infections, systemic antibiotics are taken orally or intravenously and affect the body as a whole. The choice of initial antibiotic treatment depends on several factors such as the severity of the infection, whether the patient received another antibiotic treatment, or the infection is caused by a microorganism known to be resistant to the usual antibiotics, and the patient's general health. The goal of antibiotic treatment is to stop the infection and prevent its spread.

The systemic chemotherapy approach presents the risk of generating, on the one hand, bacterial resistance, and on the other hand, side effects. Localized therapies are based on a controlled diffusion of the active anti-microbial principles incorporated in slow release devices. This therapeutic alternative makes possible to avoid general effects and obtain high active pharmacological concentrations at the targeted sites. It is essential to maintain in situ a concentration of antibiotics always greater than the minimum inhibitory concentration (MIC). Local antibiotic delivery is useful when infected sites are poorly perfused preventing proper antibiotic infusion. In addition to locally increasing the antibiotic concentration, localized therapies allow the use of antibiotics which are toxics (ototoxicity, nephrotoxicity) when used systemically.

In the art (Kanellakopoulou K et al.; Drugs. 2000. 59:1223-32), carriers used for the local delivery of antibacterial agents may be classified as non-biodegradable or biodegradable. A major representative of the former category are the polymethylmethacrylate (PMMA) beads often impregnated with gentamycin, vancomycin or tobramycin (Kanellakopoulou K et al.; Drugs. 2000 59:1223-32; and Meeker et al., 2019. J Arthroplasty. 34:1458-61). The modern use of local antibiotic in orthopaedics began with the use of polymethylmethacrylate cement impregnated with antibiotics to treat infected joint prostheses. PMMA leads to a rapid release of a high concentration of antibiotic (vancomycin, tobramycin, gentamycin, erythromycin and cefuroxime) during the first several days after implantation. The major disadvantage of this material is the need for their surgical removal at the completion of antibiotic release, which usually takes place 4 weeks after their implantation.

Examples of the latter category (biodegradable) include collagen-gentamycin sponge, apatite-wollastonite glass ceramic blocks, hydroxyapatite blocks, polylactide/polyglycolide implants and the polylactate polymers (Kanellakopoulou K et al.; Drugs. 2000. 59:1223-32). All of the above systems release antibiotics at high concentrations for the first few days without releasing any antibiotic into the systemic circulation and without producing adverse effects. The biodegradable carriers do not require surgical removal, and of those listed, the collagen-gentamycin sponge has been applied successfully over the last decade for bone infections. Collagen sponges are vector only available on the market loaded with gentamicin. They dissolve entirely in one to two weeks making them therefore unsuitable for filling cavities. In addition, their elution is extremely rapid, mainly on the first day (Sørensen T S et al; 1990. Acta Orthop Scand. 61:353-6).

Today, there is a need for a biodegradable local delivery system of antibiotics, and more particularly antimicrobial peptides that allows a delivery over an extended period while avoiding a biofilm development on the system as reported for PMMA beads. More particularly, development of novel degradable drug delivery systems (DDS) dedicated for local sustained release of peptide antibiotic participate in the response towards the emergence of multidrug resistance pathogens by reducing the systemic exposition. Use of local DDS loaded with peptides antibiotics could be an efficient tool in the treatment of infections causes by Gram-negative and Gram-positive bacteria in musculoskeletal tissues, skin and soft tissues and the eyes.

Gram-negative bacteria cause infections including pneumonia, bloodstream infections, wound or surgical site infections, and meningitis. Gram-negative bacteria are increasingly resistant to most available antibiotics. These bacteria have developed abilities to find new ways to be resistant and can pass along genetic materials that allow other bacteria to become drug-resistant (Velkov et al; 2016. Future Med Chem. 8:1017-25).

An original approach to treat infections caused by gram-negative bacteria could be based on the sustained local delivery of peptides antibiotic such peptides belonging to the polymyxin family. Polymyxins are polypeptide antibiotics discovered in 1947 and introduced into the clinic in the late 1950s. Polymyxins are cationic polypeptides that consist of a cyclic heptapeptide possessing a tripeptide side chain acylated at the N-terminal end by a fatty acid tail. Colistin (polymyxin E) and polymyxin B differ by only a single amino acid in the peptide ring, with a phenylalanine in polymyxin B and a leucine in colistin (Poirel et al; 2017. Clin Microbiol Rev. 30:557-96). Polymyxins neutralize the Gram-negative bacteria due to their high affinity for lipopolysaccharides (LPS) by means of electrostatic interactions between the negatively charged LPS (phosphate moieties of the lipid A region of LPS) and the positively charged amino-butyric residues of polymyxins. Parenteral administration of the polymyxins led to nephrotoxicity and their clinical use decreased by the 1970s. Over the last two decades there was a renewed interest in polymyxins according to the emergence of multidrug-resistant bacteria, notably among Pseudomonas aeruginosa and Acinetobacter baumannii and the absence of new antibiotics effective on these bacteria in the future. This situation should favor the rebirth of the polymyxin antibiotics, which represent an important last-line of defence against Gram-negative pathogens. Modifications of the polymyxin structure to reduce the renal toxicity but preserving the bactericidal activity have failed (Velkov et al; 2016. Future Med Chem. 8:1017-25). Until now, polymyxins were immobilized on cellulose microspheres to bind the circulating LPS for clinical blood purification during septic shock (Stegmayr. 2001. Ther Apher. 5:123-7). Polymyxins were encapsulated in PLGA microspheres to maintain a slow release of polymyxin in the blood of mice to protect them against endotoxin-induced sepsis (Nanjo et al; 2013. Journal of Infection and Chemotherapy. 19:683-90). No local DDS of polymyxins seems to have been developed until now to cure local Gram-negative bacteria. Loading of polymyxins on a tuneable degradable carrier to treat locally infections caused by Gram-negative bacteria resistant to systemic antibiotics would be an innovative anti-infective strategy.

Infections caused by multidrug-resistant Gram-positive bacteria represent a major public health burden. Gram-positive bacteria are among the most common causes of infection, and the prevalence of drug-resistant strains of Gram-positive bacteria (including methicillin-resistant Staphylococcus aureus (MRSA) and glycopeptide-resistant enterococci) is increasing (Munita et al; 2015. Clin Infect Dis. 61(Suppl 2):S48-S57). Gram-positive organisms (bacteria of the genera Staphylococcus, Streptococcus and Enterococcus) are among the most common bacterial causes of clinical infections. MRSA is a pathogen of concern due to its resistance to almost all ß-lactam antimicrobials (i.e. penicillins, cephalosporins and carbapenems). Antimicrobial peptides, in particular the glycopeptides (Vancomycin, Teicoplanin, Daptomycin, Telavancin, Dalbavancin, Oritavancin) represent a major class of molecules effective towards Gram-positive bacteria.

Glycopeptides kill Gram-positive bacteria by inhibiting the cross-linking stabilisation step in bacterial cell wall formation. They constitute an alternative to the penicillins in cases of resistance or intolerance. Vancomycin is the second most widely used antibiotic, showing antimicrobial activity against most Gram-positive germs, including MRSA. Teicoplanin (produced by the actinomycete Actinoplanes teichomyceticus) is structurally similar to vancomycin and is a complex of five closely related glycopeptides that have the same heptapeptide base and an aglycone that contains aromatic amino acids, one α-D-mannose and two N-acetyl-D-glucosamine residues, one of which is substituted with an acyl moiety composed of a fatty acid residue containing 10 or 11 carbon atoms. Teicoplanin is about five times as lipophilic as vancomycin.

Unfortunately, resistance to glycopeptides is developing among Gram-positive pathogenic bacteria, resistance to vancomycin in Staphylococcus aureus (Gardete et Tomasz; 2014. 124:2836-40) and teicoplanin in Staphylococcus epidermidis (Trueba et al; 2006. J Clin Microbiol. 44:1922-23). One way to reduce the emergence of resistance to these antibiotics of last resort is to privilege when it is possible a local therapy instead of a systemic treatment. Local delivery of glycopeptides loaded on degradable carriers represents an opportunity to treat locally serious infections while reducing the risk to create resistance which is induced during systemic treatments.

Many DDS of teicoplanin are described for local delivery in the treatment of chronic osteomyelitis. Jia et al. 2010 (Acta Biomaterialia. 6:812-19) prepared composite materials composed of borate bioactive glass and chitosan. In vitro, ≈40% of teicoplanin release occurred in 24 h and ≈70% after 7 days. In vivo after 12 weeks, the implants are efficient to treat osteomyelitis in a rabbit model by providing teicoplanin sustained release. Teicoplanin was encapsulated in poly(ethylene glycol) monomethyl ether (mPEG) and poly(lactic-co-glycolic acid) (PLGA) copolymer as a sol-gel drug delivery system for treating bone infection. Gelification of composition occurred in the target tissues and was effective to treat experimental osteomyelitis induced in rabbit (Peng et al; 2010. Biomaterials. 31:5227-36). Calcium sulfate pellets containing teicoplanin were also prepared. The in vitro drug elution lasts for 1 week (80% of release). In vivo, 6 weeks after implantation in a rabbit model of osteomyelitis, a reduction of bacterial infection and bone repair were observed (Jia et al; 2010. Antimicrob Agents Chemother. 54:170-176). Teicoplanin was encapsulated in PLGA microspheres for intra-articular implantation in rabbit. Efficient concentration of teicoplanin in synovial fluid was measured for 5 weeks (Yenice et al. 2003. Journal of Microencapsulation: 20:705-17). An in vitro sustained delivery of teicoplanin (37% in 100 h) was obtained with tri-polyphosphate cross-linked chitosan nanoparticles containing the drug (Kahdestani et al; 2020. Polym. Bull. 20). As for gelatin sponges, the teicoplanin impregnation is poor compared to vancomycin and are not considered as promising carriers for the local application of teicoplanin to infected wounds (Drognitz et al; 2006. Infection 34(1):29-34). Local delivery of teicoplanin by means of different degradable carriers is an efficient strategy in animal model to treat experimental osteomyelitis.

To locally treat infections caused by Gram-positive and Gram-negative bacteria, and to avoid the emergence of resistance due to systemic anti-biotherapy, the inventors have noticed the interest represented by the local antibiotic delivery systems. There is a need for a degradable carrier of antimicrobial peptides effective against the Gram-negative and Gram-positive germs that allows a sustained release without burst and that allows an easy and quick loading of the active ingredients. The inventors have also noticed the absence of degradable carriers for local delivery of polymyxins into wounds.

### SUMMARY OF THE INVENTION

In a first aspect, the invention relates to a composition comprising an effective amount of a peptide antibiotic, at least one hydrophilic degradable microsphere comprising a crosslinked matrix, and a pharmaceutically acceptable carrier for administration by injection, the crosslinked matrix being the crosslinked matrix being based on at least:
a) from 10 mol % to 90 mol % of a hydrophilic monomer of general formula (I):

(CH₂=CR₁)—CO-D (I)

wherein:
D is O—Z or NH—Z, with Z being —(CR₂R₃)ₘ—CH₃, —(CH₂—CH₂—O)ₘ—H, —(CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 mol % to 30 mol % of a cyclic monomer of formula (II):

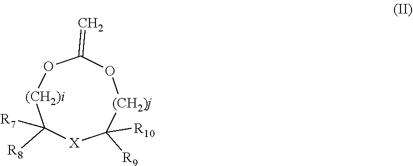

wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;

and
c) from 5 mol % to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:

(CH₂═CR₁₁)—CO—Xₙ-PEGₚ-Xₖ—CO—(CR₁₁═CH₂) (IIIa); or

W(PEGₚ-Xₙ—O—CO—(CR₁₁═CH₂))_{z} (IIIc);
wherein
   R₁₁ is independently a hydrogen atom or a (C₁-C₆)alkyl group;
   Xₙ or Xₖ is independently PLA, PGA, PLGA, PCL or PLAPCL;
   n and k are independently integers from 1 to 150;
   W is a carbon atom, a C₁-C₆-alkyl group or an ether group comprising 1 to 6 carbon atoms;
p is an integer from 1 to 100;
z is an integer from 3 to 8;

wherein mol % of components a) to c) are expressed relative to the total number of moles of compounds a), b) and c).

In a second aspect, the invention relates to the composition of the invention, for use for preventing and/or treating infectious diseases, in particular mammal infectious diseases, by local delivery.

In a third aspect, the invention relates to the hydrophilic degradable microsphere of the invention for use for locally delivering an effective amount of a peptides antibiotic to a subject in need thereof.

In a fourth aspect, the invention relates to a pharmaceutical kit comprising:

i) at least one hydrophilic degradable microsphere of the invention in association with a pharmaceutically acceptable carrier for administration by injection;

ii) an effective amount of peptides antibiotic; and

iii) optionally an injection device,

the hydrophilic degradable microsphere and the peptides antibiotic being packed separately.

### FIGURES

FIG. 1: Effect of the amount of teicoplanin in the loading medium on the drug loading on MS2. Increasing amounts of teicoplanin (10 mg up to 300 mg) in water were incubated with 0.5 mL of dry and sterile microspheres. Loading reactions were done at room temperature or at 37° C. Amount of teicoplanin (mg) loaded on 1 mL of MS (A) and efficiency (%) (B) of extemporaneous teicoplanin loading.

FIG. 2: In vitro release of teicoplanin and dalbavancin after the extemporaneous loading on the sterile and degradable microspheres of example 1. * time of degradation of microspheres in PBS during the antibiotics release at 37° C.

FIG. 3: Polymyxin B elution after extemporaneous loading on degradable MS1 and MS5 and their subsequent transfer in PBS according to example 3. * time of microsphere degradation in PBS during the elution of peptides.

FIG. 4: Polymyxin B elution after extemporaneous loading on degradable MS2, MS6, MS7 and MS10 and their subsequent transfer in PBS according to example 3. * time of microsphere degradation in PBS during the elution of peptides.

FIG. 5: Polymyxin E (colistin) elution after extemporaneous loading on degradable MS2 and MS5 and their subsequent transfer in PBS according to example 3. * time of microsphere degradation in PBS during the elution of peptides.

FIG. 6: In vitro release of human cathelicidin peptide LL-37 extemporaneously loaded on degradable MS2 of example 1. The degradation of MS2 was expressed as mg of degradation products released in PBS during the time according to the assay of pegylated compounds. *: time of degradation of MS2 in PBS during the elution of peptides.

FIG. 7: Plasma concentration of teicoplanin in rabbit after a single subcutaneous injection of degradable microspheres extemporaneously loaded with teicoplanin.

FIG. 8: Size distribution of degradable microspheres MS1 & MS2.

### DETAILED DESCRIPTION OF THE INVENTION

The inventors have surprisingly found that degradable hydrophilic microspheres composed of a crosslinked hydrogel display strong affinity for peptide antibiotics, in particular for both polymyxins and teicoplanin peptides, allowing their efficient loading in few minutes onto preformed microspheres and their subsequent sustained release.

The present invention offers the possibility to simplify the process for the local delivery of different antimicrobial peptides in infected tissues, i.e by simply mixing antimicrobial peptides in solution in water with preformed degradable microspheres, a simpler procedure than the preparation of PMMA beads impregnated with antibiotics (Meeker et al., 2019. J Arthroplasty 34:1458-61)

The inventors have discovered hydrophilic degradable microspheres that may be used as biocompatible drug carrier for local delivery and that present affinity with polycationic peptides which may have a fatty acid tail, including polymyxins, and glycopeptide antibiotics having a fatty acid tail, including teicoplanin.

The inventors have thus discovered a local delivery system of antimicrobial peptides. The delivery system is free of organic solvent with a tuneable degradation time ranging from day to months, that is easy to load (in water, at room temperature in few minutes), that allows a complete drug release and that avoids intense inflammatory reaction and immediate burst.

The local delivery system of the invention allows an extended and local release of antimicrobial peptides. Such a delivery avoids the release of any antibiotic in the systemic circulation and thus the adverse effects while allowing an extended duration of release without the need of several administration and without surgical removal of the delivery system.

### Definition

As used herein, the expression “matrix based on” means a matrix comprising a mixture of at least components (a) to (c) and/or a matrix resulting from the reaction, in particular from the polymerization, between at least components (a) to (c). Hence, components (a) to (c) can be seen as the starting components that are used for the polymerization (e.g. heterogenous medium polymerization) of the matrix.

The expression “reaction mixture” as used herein designates the polymerisation medium including any components taking part to the polymerisation. The reaction mixture typically comprises at least components a), b), c) as defined in the claims and in this description, optionally a polymerization initiator such as, for example, t-butyl peroxide, benzoyl peroxide, azobiscyanovaleric acid (also called 4,4′-azobis(4-cyanopentanoic acid)), AIBN (azobisisobutyronitrile), or 1,1′-azobis(cyclohexane carbonitrile) or optionally one or more photo-initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenyl acetophenone (Irgacure®, 24650-42-8) or 2-methyl-4′-(methylthio)-2-morpholinopropiophenone (Irgacure®, 71868-10-5), and at least one solvent, preferably a solvent mixture comprising an aqueous solvent and an organic solvent such as an apolar aprotic solvent, for example a water/toluene mixture and optionally any suitable components as described herein (e.g. stabilizer such as polyvinyl alcohol).

Thus, in the present description, expressions such as “the [starting component X] is added to the reaction mixture in an amount of between YY % and YYYY %” and “the cross-linked matrix is based on [starting component X] in an amount of between YY % and YYYY %” are interpreted in a similar manner. Similarly, expressions such as “the reaction mixture comprises at least [starting component X]” and “the cross-linked matrix is based on at least [starting component X]” are interpreted in a similar manner.

In the context of the invention, “organic phase” of the reaction mixture means the phase comprising the organic solvent and the compounds soluble in said organic solvent, in particular the monomers, and the polymerization initiator.

As used herein, the terms “(C_{X}-C_{Y})alkyl group” mean a saturated monovalent hydrocarbon chain, linear or branched, containing X to Y carbon atoms, X and Y being integers between 1 and 36, preferably between 1 and 18, in particular between 1 and 6. Examples are methyl, ethyl, propyl, iso-propyl, butyl, iso-butyl, sec-butyl, tert-butyl, pentyl or hexyl groups.

As used herein, the terms “aryl group” and “(C_{X}-C_{Y})aryl” mean an aromatic hydrocarbon group, preferably having X to Y carbon atoms, X and Y being integers between 6 and 36, preferably between 6 and 18, in particular between 6 and 10. The aryl group may be monocyclic or polycyclic (fused rings). Examples are phenyl or naphtyl groups.

As used herein, the terms “partition coefficient P” mean the ratio of concentrations of a compound in a mixture of two immiscible solvents at equilibrium: water and 1-octanol. This ratio is therefore a comparison of the solubilities of the solute in these two liquids. Hence the octanol/water partition coefficient measures how hydrophilic (octanol/water ratio<1) or hydrophobic (octanol/water>1) a compound is. Partition coefficient P may be determined by measuring the solubilities of the compound in water and in 1-octanol and by calculating the ratio solubility in octanol/solubility in water. Partition P may also be determined in silico using Chemicalize provided by ChemAxon.

As used herein, the hydrophobic/hydrophilic balance R of the degradable crosslinkers is quantified by the ratio of the number of hydrophobic units to the number of hydrophilic units according the following equation:

$$R = \frac{{N}_{hydrophobic units}}{{N}_{hydrophilic units}}$$

with N being an integer and representing the number of unit(s).

For example, for the crosslinkers that can be used in the present invention, R is:

$$R = \frac{{N}_{CHlactide} + {N}_{CH 3 lactide} + {N}_{CH 2 glycolide} + 5 \times {N}_{CH 2 caprolactone}}{{N}_{EO unit}}$$

with N being an integer and representing the number of unit(s).

As used herein, the terms “degradable microsphere” mean that the microsphere is degraded or cleaved by hydrolysis in a mixture of degradation products composed of low-molecular-weight compounds and water-soluble polymer chains having molecular weights below the threshold for renal filtration of 50 kg·mol⁻¹.

As used herein, the expression “hydrophilic degradable microsphere” means a degradable microsphere containing from 10% to 90% of a hydrophilic monomer which allows a good compatibility with the aqueous media and a low adhesion to solid surface (syringes, needles, catheters).

As used herein, the expression “between X and Y” (wherein X and Y are numerical value) means a range of numerical values in which the limits X and Y are inclusive.

As used herein, the expression “immediate release (IR)” of an active ingredient means the rapid release of the active ingredient from the formulation to the location of delivery as soon as the formulation is administered.

As used herein, the expression “extended-release” of an active ingredient means either the “sustained-release (SR)” or the “controlled-release (CR)” of active ingredients from the formulation to the location of delivery at a predetermined rate for an extended period of time and maintaining a constant active ingredient level for this period of time with minimum side effects. The controlled-release (CR) differs from the sustained-release (SR) in that CR maintains drug release over a sustained period at a constant rate whereas SR maintains drug release over a sustained period but not at a constant rate.

As used herein, the expression “sustained-release” of an active ingredient means an extended-release (as defined above) of an active ingredient from the formulation to the location of delivery in order to maintain for a certain predetermined time the drug in tissue of interest at therapeutic concentrations by means of an initial dose portion.

As used herein, the expression “controlled-release (CR)” of an active ingredient means an extended-release (as defined above) of an active ingredient from the formulation to the location of delivery that provides some control of temporal or spatial nature, or both.

As used herein, the term “pharmaceutically acceptable” is intended to mean what is useful to the preparation of a pharmaceutical composition, and what is generally safe and non toxic, for a pharmaceutical use.

As used herein, the terms «pharmaceutically acceptable salt» mean a salt of a compound which is pharmaceutically acceptable, as defined above, and which possesses the pharmacological activity of the corresponding compound. Such salts comprise:

(1) hydrates and solvates,

(2) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and

(3) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Molar percentage is abbreviated herein as mol %.

Microsphere

According to the present invention, the hydrophilic degradable microsphere comprises a crosslinked matrix that is based on at least, preferably that results from the polymerization of, at least the following components:
a) from 10 to 90 mol % of a hydrophilic monomer of general formula (1):

(CH₂═CR₁)—CO-D (I)

wherein:
D is O—Z or NH—Z, with Z being —(CR₂R₃)ₘ—CH₃, —(CH₂—CH₂—O)ₘ—H, —(CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 to 30 mol % of a cyclic monomer of formula (II):

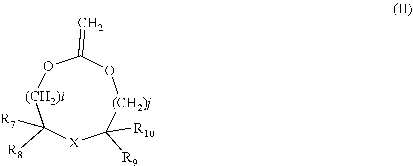

wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;

and
c) from 5 to 90 mol % of a degradable block copolymer cross-linker, wherein the degradable block copolymer crosslinker is linear or star-shaped and presents (CH₂═(CR_{I}))-groups at all its extremities, each R₁₁ being independently of one another hydrogen atom or a (C₁-C₆)alkyl group, and wherein the degradable block copolymer crosslinker has a partition coefficient P between −3 and 11.2, typically between 0.5 and 11.2, or a hydrophobic/hydrophilic balance R between 1 and 20;

wherein mol % of components a) to c) are expressed relative to the total number of moles of compounds a), b) and c).

The partition coefficient P is determined in silico using Chemicalize provided by ChemAxon.

When the hydrophilic degradable microsphere comprises a crosslinked matrix that is based on further monomers (see monomer e) below), the mol % of components a) to c) are expressed relative to the total number of moles of compounds a), b), c) and e).

The terms “hydrophilic monomer” mean a monomer having a high affinity for water, i.e. tending to dissolve in water, to mix with water, to be wetted by water, or that gives rises to a polymer capable of swelling in water after polymerization.

The block copolymer cross-linker is a degradable block copolymer cross-linker, i.e. a polymer with linear (or radial) arrangement of different blocks joined by covalent bond. In a degradable block copolymer the covalent bond are degradable such as ester bonds, amide bonds, anhydride bond, urea bond or polysaccharidic bond and here specifically ester bonds.

When X is a single bond, it is meant that the carbon atoms bearing R₇, R₈, R₉ and R₁₀ groups are directly linked via a single bond.

The hydrophilic degradable microsphere is a swellable degradable (i.e. hydrolyzable) cross-linked polymer in the form of spherical particle having a diameter after swelling in physiological saline solution (i.e. normal saline solution) ranging between 20 μm and 1200 μm. In particular the polymer of the invention is constituted of at least one chain of polymerized monomers a), b) and c) as defined above.

In the context of the invention, a polymer is swellable if it has the capacity to absorb liquids, in particular water. The expression “size after swelling” means thus that the size of the microspheres is considered after the polymerization and sterilization steps that take place during their preparation.

Advantageously, the microsphere of the invention has a diameter after swelling in physiological saline solution (i.e. normal saline solution) of between 20 μm and 100 μm, 40 μm and 150 μm, 100 μm and 300 μm, 300 μm and 500 μm, 500 μm and 700 μm, 700 μm and 900 μm or 900 μm and 1200 μm, as determined by optical microscopy. Microspheres are advantageously small enough in diameter to be injected through needles, catheters or microcatheters with internal diameters ranging from a few hundred micrometres to more than one millimetre.

The hydrophilic monomer a) is of general formula (I):

(CH₂═CR₁)—CO-D (I)

wherein:
D is O—Z or NH—Z, with Z being —(CR₂R₃)ₘ—CH₃, —(CH₂—CH₂—O)ₘ—H, (CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group.

Advantageously, the hydrophilic monomer a) is selected from the group consisting of sec-butyl acrylate, n-butyl acrylate, t-butyl acrylate, t-butyl methacrylate, methylmethacrylate, N-dimethyl-aminoethyl(methyl)acrylate, N,N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate; advantageously acrylate terminated poly(ethylene glycol), methacrylate terminated poly(ethylene glycol), methoxy poly(ethylene glycol) methacrylate, butoxy poly(ethylene glycol) methacrylate.

In some embodiments, in the formula (I), when Z is —(CR₂R₃)ₘ—CH₃ or —(CR₂R₃)ₘ—OH, m is preferably an integer from 1 to 6.

In some embodiments, in the formula (I), when Z is —(CR₂R₃)ₘ—CH₃, Z is preferably a C₁-C₆-alkyl group.

In some embodiments, in the formula (I), when Z is —(CR₂R₃)ₘ—OH, R₂ and R₃ are preferably hydrogen and m is an integer from 1 to 6.

In some embodiments, the hydrophilic monomer a) is of general formula (1):

(CH₂═CR₁)—CO-D (I)

wherein:
D is O—Z, with Z being —(CH₂—CH₂—O)ₘ—H or —(CH₂—CH₂—O)ₘ—CH₃, with m being an integer from 1 to 30;
R₁ is hydrogen atom or a (C₁-C₆)alkyl group, preferably a methyl.

More advantageously, the hydrophilic monomer a) is poly(ethylene glycol) methyl ether methacrylate (m-PEGMA).

The amount of hydrophilic monomer a) typically ranges from 10 mol % to 90 mol %, preferably from 30 mol % to 85 mol %, more preferably from 30 mol % to 80 mol %, relative to the total number of moles of components a), b) and c) (or relative to the total number of moles of components a), b), c) and e) when e) is present—see below).

Component b) is a cyclic monomer of formula (II) as defined above, wherein:
R₇, R₈, R₉ and R₁₀ are, independently of one another, hydrogen atom, a (C₁-C₆)alkyl group or an aryl group;
i and j are independently of one another an integer chosen between 0 and 2;
X is a single bond or an oxygen atom.

Advantageously, component b) is a cyclic monomer of formula (II) as defined above, wherein:
R₇, R₈, R₉ and R₁₀ are, independently of one another, hydrogen atom or a (C₅-C₇)aryl group;
i and j are independently of one another an integer chosen between 0 and 2;
X is a single bond or an oxygen atom.

Advantageously, component b) is a cyclic monomer of formula (II) as defined above, wherein:
R₇, R₈, R₉ and R₁₀ are, independently of one another, hydrogen atom or a (C₅-C₇)aryl group;
i and j are independently of one another an integer chosen between 0 and 1;
X is a single bond or an oxygen atom.

Advantageously, the component b) is selected from the group consisting of 2-methylene-1,3-dioxolane, 2-methylene-1,3-dioxane, 2-methylene-1,3-dioxepane, 2-methylene-1,3,6-trioxocane and derivatives thereof, in particular benzo derivatives and phenyl substituted derivatives, advantageously from the group consisting of 2-methylene-1,3-dioxolane, 2-methylene-1,3-dioxane, 2-methylene-1,3-dioxepane, 2-methylene-4-phenyl-1,3-dioxolane, 2-methylene-1,3,6-trioxocane and 5,6-benzo-2-methylene-1,3dioxepane, more advantageously from the group consisting of 2-methylene-1,3-dioxepane, 5,6-benzo-2-methylene-1,3-dioxepane and 2-methylene-1,3,6-trioxocane. More advantageously, the component b) is 2-methylene-1,3-dioxepane or 2-methylene-1,3,6-trioxocane.

The amount of component b) typically ranges from 0.1 mol % to 30 mol %, preferably from 1 mol % to 20 mol %, and in particular from 1 mol % to 10 mol %, relative to the total number of moles of components a), b) and c) (or relative to the total number of moles of components a), b), c) and e) when e) is present—see below). In some embodiments, the amount of component b) is about 10 mol %.

The cyclic monomer b) of general formula (II) is advantageously present in the reaction mixture in an amount ranging from 0.1 mol % to 30 mol %, preferably from 1 mol % to 20 mol %, and in particular from 5 mol % to 15 mol % or from 1 mol % to 10 mol %, relative to the total number of moles of components a), b) and c). In some embodiments, the amount of component b) is about 10 mol %.

Component c) is a degradable block copolymer crosslinker, wherein the degradable block copolymer crosslinker is linear or star-shaped and presents (CH₂═(CR₁₁))-groups at all its extremities, each R₁₁ being independently of one another hydrogen atom or a (C₁-C₆)alkyl group.

The degradable block copolymer crosslinker has a partition coefficient P of between −3 and 11.20, typically between 0.5 and 11.20, advantageously between 2.00 and 9.00; or the degradable block copolymer crosslinker has a hydrophobic/hydrophilic balance R between 1 and 20, advantageously between 3 and 15.

The hydrophobicity of the crosslinker and its concentration, and thus the partition coefficient P of the crosslinker, influences the release of the peptide antibiotics. Thus, when the partition coefficient P increases, the delivery time of peptide antibiotic, in particular of polycationic peptide antibiotics also increases. The concentration of the crosslinker also influences the release of peptide antibiotics, in particular of hydrophobic glycopeptide antibiotics, high concentration of crosslinker prevents the immediate release of a large part of the peptide antibiotics, and lengthens the release time.

As intended therein, the expression “copolymer cross-linker” is intended to mean that the copolymer contains a functional group containing a double bond at least two of its extremities in order to link together several polymer chains.

The cross-linker c) as defined above is linear or star-shaped (advantageously from 3 to 8 arms) and it presents (CH₂═(CR₁₁))-groups at all its extremities, each R₁₁ being independently of one another hydrogen atom or a (C₁-C₆)alkyl group, preferably a methyl group. Advantageously, the crosslinker c) presents (CH₂═(CR₁₁))—CO— at all its extremities, each R₁₁ being independently of one another hydrogen atom or a (C₁-C₆)alkyl group, preferably a methyl group. Advantageously, the R₁₁ are identical and are H or a (C₁-C₆)alkyl group, preferably a methyl group.

Advantageously, the cross-linker c) as defined above is linear and it presents (CH₂═(CR₁₁))-groups at both its extremities, each R₁₁ being independently of one another hydrogen atom or a (C₁-C₆)alkyl group. Advantageously, the crosslinker c) presents (CH₂═(CR₁₁))—CO— groups at both its extremities, each R₁₁ being independently of one another hydrogen atom or a (C₁-C₆)alkyl group, preferably a methyl group. Advantageously, the R₁₁ are identical and are H or a (C₁-C₆)alkyl group, preferably a methyl group.

The crosslinker c) is of general formula (IIIa) or (IIIc) as follows:

(CH₂═CR₁₁)—CO—Xₙ-PEG_{P}-Xₖ—CO—(CR₁₁═CH₂) (IIIa);

W-(PEGₚ-Xₙ—O—CO—(CR₁₁═CH₂))_{z} (IIIc);

wherein:
each R₁₁ is independently of one another hydrogen atom or a (C₁-C₆)alkyl group;
X independently represents PLA, PGA, PLGA, PCL or PLAPCL;
n, k and p respectively represent the degree of polymerization of X, and PEG, n and k independently being integers from 1 to 150, and p being an integer from 1 to 100;
W is a carbon atom, a C₁-C₆-alkyl group (preferably a C₁-C₃-alkyl) or an ether group comprising 1 to 6 carbon atoms, preferably 1 to 3 carbon atoms;
z is an integer from 3 to 8.

Crosslinker c) of formula (IIIc) is a star-shaped polymer, i.e., a polymer consisting of several linear chains (also designated arms) connected a central core. In the crosslinker of formula (IIIc), W is the core of the star-shaped polymer and -(PEGₚ-Xₙ—O—CO—(CR₁₁═CH₂) is an arm of the star-shaped polymer with z being the number of arms.

Advantageously, when the crosslinker c) is of general formula (IIIc), n may be identical or different in each arm of the PEG.

In the context of the invention, the abbreviations used herein have the following meaning:

| Abb. | Name | | Formula |
|---|---|---|---|
| PEG | polyethylene glycol | PEGₚ | 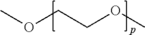 |
| PLA | poly-lactic acid (also named poly-lactide) | PLA_{n or k} | 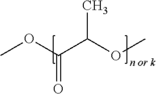 |
| PGA | poly-glycolic acid (also named poly-glycolide) | PGA_{n or k} | 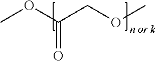 |
| PLGA | poly-lactic-glycolic acid The copolymer comprises both lactide and glycolide units, the degree of polymerization is the sum of the number of lactide and glycolide units | PLGA_{n or k} | 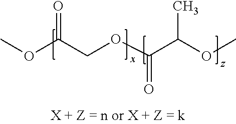 |
| PCL | poly(caprolactone) | PCL_{n or k} | 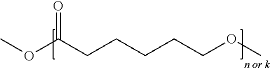 |
| PLAPCL | poly-lactic acid poly- caprolactone The copolymer comprises both lactide and caprolactone units, the degree of polymerization is the sum of the number of | PLAPCL_{n or k} | 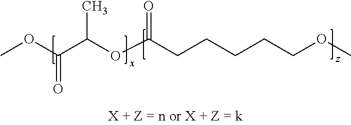 |
| | lactide and caprolactone | | |
| | units | | |

In the above table, n, p and k have the values disclosed herein.

In the above formula (IIIa), p is preferably an integer from 1 to 25, preferably from 2 to 15.

In the above formula (IIIc), p is preferably an integer from 1 to 16.

Advantageously, the crosslinker c) is of general formula (IIIa) or (IIIc), in particular (IIIa), as defined above, wherein X represents PLAPCL or PCL. More advantageously, the crosslinker c) is of general formula (IIIa) or (IIIc), in particular (IIIa), wherein X represents PCL.

Advantageously, the crosslinker c) is of general formula (IIIa) or (IIIc), in particular (IIIa), as defined above, wherein n and k independently being integers from 1 to 150, preferably from 1 to 20, more preferably from 1 to 10, even more preferably from 4 to 7. Preferably n+k ranges from 5 to 15 or from 8 to 14 and p is an integer from 1 to 100, preferably from 1 to 20.

Advantageously, the crosslinker c) is of general formula (IIIa) or (IIIc), in particular (IIIa), as defined above, wherein the R₁₁ are identical and are H or a (C₁-C₆)alkyl group.

Advantageously, the crosslinker c) is selected from the group consisting of compounds of general formula (IIIa) or (IIIc), in particular (IIIa), as defined above, wherein:
X=PLA, n+k=12 and p=13 (such as PEG₁₃-PLA₁₂ wherein R₁₁ is methyl);
X=PLAPCL, n+k=10 and p=13 (such as PEG₁₃-PLA₈-PCL₂ or PEG₁₃-PLA₇-PCL₃, wherein R₁₁ is methyl);
X=PLAPCL, n+k=9 and p=13 (such as PEG₁₃-PLA₄-PCL₅, wherein R₁₁ is methyl);
X=PLAPCL, n+k=8 and p=13 (such as PEG₁₃-PLA₂-PCL₆, wherein R₁₁ is methyl);
X=PCL; n+k=8 and p=13 (such as PEG₁₃-PCL₈, wherein R₁₁ is methyl);
X=PLGA; n+k=12 and p=13 (such as PEG₁₃-PLGA₁₂, wherein R₁₁ is methyl);
X=PCL, n+k=10 and p=4 (such as PEG₄-PCL₁₀, wherein R₁₁ is methyl); or
X=PCL, n+k=12 and p=2 (such as PEG₂-PCL₁₂, wherein R₁₁ is methyl).

In these embodiments, R₁₁ is preferably hydrogen or methyl.

In some embodiments, the crosslinker c) is a compound of general formula (IIIc), as defined above, wherein p is 7, X=PLAPCL, n=10, z is 3 with R₁₁ being preferably hydrogen or methyl (such as PEG 3-arm-PLA₇-PCL₃, wherein R₁₁ is methyl).

In some embodiments, the crosslinker c) is selected from the group consisting of compounds of general formula (IIIa) or (IIIc), in particular (IIIa), as defined above, wherein:
X=PLA, n+k=12 and p=13 (such as PEG₁₃-PLA₁₂, wherein R₁₁ is methyl); or
X=PLAPCL, n+k=10 and p=13 (such as PEG₁₃-PLA₈-PCL₂ or PEG₁₃-PLA₇-PCL₃, wherein R₁₁ is methyl); or
X=PLGA; n+k=12 and p=13 (such as PEG₁₃-PLGA₁₂, wherein R₁₁ is methyl); or
X=PCL, n+k=12 and p=2 (such as PEG₂-PCL₁₂, wherein R₁₁ is methyl).

In these embodiments, R₁₁ is preferably hydrogen or methyl.

Within the definitions of the crosslinker c) above, the polyethylene glycol (PEG) has a number average molecular weight (Mn) of 100 to 10 000 g/mol, preferably 100 to 2 000 g/mol, more preferably 100 to 1 000 g/mol

The amount of crosslinker c) typically ranges from 5 mol % to 90 mol %, preferably from 5 mol % to 60 mol %, relative to the total number of moles of components a), b) and c) (and e) when present, see below).

The crosslinker c) is advantageously present in the reaction mixture in an amount ranging from 5 mol % to 90 mol %, preferably from 5 mol % to 60 mol %, relative to the total number of moles of components a), b) and c) (and e) when present).

Advantageously, when the composition of the invention comprises glycopeptides antibiotic such as teicoplanin as peptide antibiotic, the concentration of the crosslinker influences its release. For amount greater than 10 mol %, an optimal release of the glycopeptides antibiotic such as teicoplanin is achieved, in particular because it prevents the immediate release of a large part of the glycopeptides antibiotic. Therefore, when the composition of the invention comprises glycopeptides antibiotic such as teicoplanin as peptide antibiotic, the crosslinker c) is advantageously present in the reaction mixture in an amount of between 5 mol % and 90 mol %, preferably from between 5 mol % and 60 mol %, and in particular between 10 mol % and 60 mol %, relative to the total number of moles of the monomers (components a), b) and c) and e) when present).

Advantageously, when the composition of the invention comprises polycationic peptides antibiotic such as polymyxin B as peptides antibiotic, the hydrophobicity of the crosslinker and thus the partition coefficient P of the crosslinker or the hydrophobic/hydrophilic balance R influences its release. For partition coefficient P of between 0.50 and 11.20, advantageously between 2.00 and 9.00, or hydrophobic/hydrophilic balance R between 1 and 20, preferentially between 3 and 15, an optimal release of the polycationic peptides antibiotic such as polymyxin B is achieved, in particular because it prevents the immediate release of a large part of the polycationic peptides antibiotic.

The crosslinked matrix of the hydrophilic degradable microsphere is advantageously further based on a chain transfer agent d), preferably results from the polymerization of components a), b) and c) in presence of a chain transfer agent d).

For the purposes of this invention, “transfer agent” means a chemical compound having at least one weak chemical bond. This agent reacts with the radical site of a growing polymer chain and interrupts the growth of the chain. In the chain transfer process, the radical is temporarily transferred to the transfer agent which restarts growth by transferring the radical to another polymer or monomer.

Advantageously, the chain transfer agent d) is selected from the group consisting of monofunctional or polyfunctional thiols, alkyl halides, transition metal salts or complexes and other compounds known to be active in free radical chain transfer processes such as 2,4-diphenyl-4-methyl-1-pentene. More advantageously, the chain transfer agent is a cycloaliphatic or aliphatic, thiol preferably having from 2 to 24 carbon atoms, more preferably between 2 an 12 carbon atoms, and having or not a further functional group selected from the groups amino, hydroxy and carboxy.

Advantageously, the chain transfer agent d) is selected from the group consisting of thioglycolic acid, 2-mercaptoethanol, dodecane thiol and hexane thiol.

The amount of chain transfer agent d) typically ranges from 0.1 to 10 mol %, preferably from 2 to 5 mol %, relative to the number of moles of hydrophilic monomer a).

The chain transfer agent d) is advantageously present in the reaction mixture in an amount of, for example, from 0.1 to 10 mol %, preferably from 2 to 5 mol %, relative to the number of moles of hydrophilic monomer a).

In a particular aspect of the invention, the crosslinked matrix is only based on starting components a), b), c) and optionally d), as defined above and in the contents abovementioned, no other starting component are thus added to the reaction medium. It is thus clear that the sum of the above-mentioned contents of monomers (components (a), (b) and (c)) must be equal to 100%.

In some embodiments, the crosslinked matrix is advantageously further based on, preferably results from the polymerization of, at least one ionised or ionisable monomer e) of general formula (V):

(CH₂═CR₁₂)-M-E (V),

wherein:
R₁₂ is hydrogen atom or a (C₁-C₆)alkyl group;
M is a single bond or a divalent radical having 1 to 20 carbon atoms, advantageously a single bond;
E is a ionised or ionisable group being advantageously selected from the group consisting of —COOH, —COO⁻, —SO₃H, —SO₃⁻, —PO₄H₂, —PO₄H⁻, —PO₄²⁻, —NR₁₃R₁₄, and —NR₁₅R₁₆R₁₇*; R₁₃, R₁₄, R₁₅, R₁₆ et R₁₇ being independently of one another hydrogen atom or a (C₁-C₆)alkyl group.

In the context of the invention, an ionised or ionisable group is understood to be a group which is charged or which may be in charged form (in the form of an ion), i.e. which carries at least one positive or negative charge, depending on the pH of the medium. For example, the COOH group may be ionised in the COO⁻ form, and the NH₂ group may be ionised in the form of NH₃*.

The introduction of an ionised or ionisable monomer into the reaction media makes it possible to increase the hydrophilicity of the resulting microspheres, thereby increasing the swelling rate of said microspheres, further facilitating their injection via catheters and microcatheters. In addition, the presence of an ionised or ionisable monomer improves the loading of active substances into the microsphere.

In an advantageous embodiment, the ionised or ionisable monomer e) is a cationic monomer, advantageously selected from the group consisting of 2-(methacryloyloxy)ethyl phosphorylcholine, 2-(dimethylamino)ethyl (meth)acrylate, 2-(diethylamino)ethyl (meth)acrylate and 2-((meth)acryloyloxy)ethyl] trimethylammonium chloride, more advantageously the cationic monomer is diethylaminoethyl (meth)acrylate. Advantageously, the ionised or ionisable e) is present in the reaction mixture in an amount of between 0% and 30% by mole, advantageously between 1% and 30% by mole, preferably from between 10% and 20% or 15% by mole, relative to the total number of moles of the monomers (components a)+b)+c)+e)). It is thus clear that in such a case the sum of the above-mentioned contents of monomers (components (a), (b) and (c) and (e)) must be equal to 100%.

In another advantageous embodiment, the ionised or ionisable monomer e) is an anionic monomer advantageously selected from the group consisting of acrylic acid, methacrylic acid, 2-carboxyethyl acrylate, 2-carboxyethyl acrylate oligomers, 3-sulfopropyl (meth)acrylate potassium salt and 2-(methacryloyloxy)ethyl]dimethyl-(3-sulfopropyl)ammonium hydroxide, more advantageously, the anionic monomer is acrylic acid or methacrylic acid. Advantageously, the ionised or ionisable monomer e) is present in the reaction mixture in an amount of between 0% and 50% by mole, advantageously between 1% and 50% by mole, preferably from between 10% and 30% by mole, relative to the total number of moles of the monomers (components a)+b)+c)+e)). It is thus clear that in such a case the sum of the above-mentioned contents of monomers (components (a), (b) and (c) and (e)) must be equal to 100%.

Advantageously, the ionised or ionisable monomer e) is acrylic acid and is advantageously present in the reaction mixture in an amount that ranges from 0% to 30% by mole, advantageously from 1% and 30% by mole, preferably from 10% to 15% by mole, relative to the total number of moles of the monomers (components a)+b)+c)+e)). It is thus clear that in such a case the sum of the above-mentioned contents of monomers (components (a), (b) and (c) and (e)) must be equal to 100%.

In some embodiments, the hydrophilic degradable microsphere comprises a crosslinked matrix that is based on at least, preferably that results from the polymerization of, the following components: a) from 10 to 90 mol % of a hydrophilic monomer of general formula (I):

(CH₂═CR₁)—CO-D (I)

wherein:
D is O—Z, Z being —(CH₂—CH₂—O)ₘ—H or —(CH₂—CH₂—O)ₘ—CH₃, with m being an integer from 1 to 30;
R₁ is hydrogen atom or a (C₁-C₆)alkyl group, preferably a methyl; preferably m-PEGMA,
b) from 0.1 to 30 mol % of a cyclic monomer of formula (II):

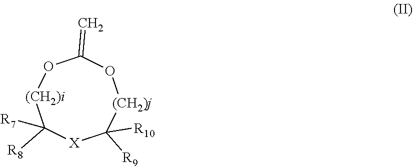

wherein:
R₇, R₈, R₉ and R₁₀ are, independently of one another, hydrogen atom or a (C₅-C₇)aryl group;
i and j are independently of one another an integer chosen between 0 and 1;
X is a single bond or an oxygen atom;
preferably 2-methylene-1,3-dioxepane;

and
c) from 5 to 90 mol % of a degradable block copolymer cross-linker of formula:

(CH₂═CR₁₁)—CO—Xₙ-PEGₚ-Xₖ—CO—(CR₁₁═CH₂) (IIIa), or

W(PEGₚ-Xₙ—O—CO—(CR₁₁═CH₂))_{z}(IIIc);
wherein R₁₁, X, W, n, p, k, z are as disclosed herein,
preferably wherein
   R₁₁ is independently of one another hydrogen atom or a (C₁-C₆)alkyl group;
   X=PLA, n+k=12 and p=13 (such as PEG₁₃-PLA₁₂ wherein R₁₁ is methyl); or
   X=PLAPCL, n+k=10 and p=13 (such as PEG₁₃-PLA₈-PCL₂ or PEG₁₃-PLA₇-PCL₃, wherein R₁₁ is methyl); or
   X=PLAPCL, n+k=9 and p=13 (such as PEG₁₃-PLA₄-PCL₅, wherein R₁₁ is methyl); or
   X=PLAPCL, n+k=8 and p=13 (such as PEG₁₃-PLA₂-PCL₆, wherein R₁₁ is methyl); or
   X=PLGA; n+k=12 and p=13 (such as PEG₁₃-PLGA₁₂, wherein R₁₁ is methyl); or
   X=PCL; n+k=8 and p=13 (such as PEG₁₃-PCL₈, wherein R₁₁ is methyl); or
   X=PCL, n+k=10 and p=4 (such as PEG₄-PCL₁₀, wherein R₁₁ is methyl); or
   X=PCL, n+k=12 and p=2 (such as PEG₂-PCL₁₂, wherein R₁₁ is methyl);
and wherein the degradable block copolymer crosslinker has a partition coefficient P between −3 and 11.2, typically between 0.5 and 11.2, or a hydrophobic/hydrophilic balance R between 1 and 20;

wherein mol % of components a) to c) are expressed relative to the total number of moles of compounds a), b) and c).

When the hydrophilic degradable microsphere comprises a crosslinked matrix that is based on further monomers (see monomer e) above), the mol % of components a) to c) are expressed relative to the total number of moles of compounds a), b), c) and e).

The amounts of components a), b) and c) may be as disclosed herein.

The microsphere of the invention can be readily synthesized by numerous methods well-known to the one skilled in the art. By way of example, the microsphere of the invention can be obtained by direct or inverse suspension polymerization as described below and, in the Examples, or by microfluidic.

A direct suspension may proceed as follows:

(1) stirring or agitating a mixture comprising
(i) at least the starting components a), b) and c) as defined above;
(ii) a polymerization initiator present in amounts ranging from 0.1 to approximately 2 parts per weight per 100 parts by weight of the monomers;
iii) a surfactant in an amount no greater than about 5 parts by weight per 100 parts by weight of the aqueous solution, preferably no greater than about 3 parts by weight and most preferably in the range of 0.5 to 1.5 parts by weight;
(iv) a salt in an amount no greater than about 10 parts by weight per 100 parts by weight of the aqueous solution, preferably no greater than about 5 parts by weight and most preferably in the range of 1 to 4 parts by weight; and (v) water to form an oil in water suspension;

and

(2) polymerizing the starting components.

In such a direct suspension polymerization, the surfactant may be selected from the group consisting of hydroxyethylcellulose, polyvinyl alcohol (PVA), polyvinylpyrrolidone, polyethylene oxide, polyethylene glycol and polysorbate 20 (Tween® 20).

An inverse suspension may proceed as follows:

(1) stirring or agitating a mixture comprising:
(i) at least the starting components a), b) and c) as defined above;
(ii) a polymerization initiator present in amounts ranging from 0.1 to approximately 2 parts per weight per 100 parts by weight of the monomers;
(iii) a surfactant in an amount no greater than about 5 parts by weight per 100 parts by weight of the monomers, preferably no greater than about 3 parts by weight and most preferably in the range of 0.5 to 1.5 parts by weight; and
(iv) oil to form a water in oil suspension;

and

(2) polymerizing the starting components.

In such a reverse suspension process, the surfactant may be selected from the group consisting of sorbitan esters such as sorbitan monolaurate (Span® 20), sorbitan monopalmitate (Span® 40), sorbitan monooleate (Span® 80), and sorbitan trioleate (Span® 85), hydroxyethyl cellulose, mixture of glyceryl stearate and PEG stearate (Arlacel®) and cellulose acetate.

In the above processes, the polymerization initiator may include t-butyl peroxide, benzoyl peroxide, azobiscyanovaleric acid (also known as 4,4′-azobis(4-cyanopentanoic acid)), AIBN (azobisisobutyronitrile), or 1,1′ azobis(cyclohexane carbonitrile) or one or more thermal initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenyl acetophenone (Irgacure®, 24650-42-8) or 2-methyl-4′-(methylthio)-2-morpholinopropiophenone (Irgacure®, 71868-10-5).

Further, the oil may be selected from paraffin oil, silicone oil and organic solvents such as hexane, cyclohexane, ethyl acetate or butyl acetate.

Loading may proceed by numerous methods well-known to one of skill in the art such as passive adsorption (swelling of the polymer into a drug solution) or by ionic interaction.

In order to increase the drug loading and control the rate of drug release, a concept consists to introduce certain chemical moieties into the polymer backbone that are capable of interacting with the drug via non covalent interactions. Examples of such interactions include electrostatic interactions (described after), hydrophobic interactions, r-r stacking, and hydrogen bonding, among others.

Drug

The composition comprises an effective amount of a peptide antibiotic. Peptide antibiotics, also named antimicrobial peptides, are a chemically diverse class of anti-infective and antitumor antibiotics containing non-protein polypeptide chains. Antimicrobial peptide antibiotics include in particular glycopeptide antibiotics, and polycationic peptide antibiotics.

Advantageously, the antimicrobial peptide antibiotics are glycopeptide antibiotics, in particular hydrophobic glycopeptide antibiotics, and polycationic peptide antibiotics, in particular polycationic peptides, cyclic or linear, with or without a fatty acid tail.

In the context of the invention, a “polycationic peptide” may be a cyclic peptide soluble in water, i.e. having a partition coefficient P between −2.4 (polymyxin E-colistin) and −0.9 (polymyxin B). The polymyxin scaffold contains a hydrophobic tail, namely a methyl-octanamide chain covalently bound terminal diaminobutyric acid.

In the context of the invention, a “polycationic peptide” may also be a linear peptide soluble in water produced by different organisms (bacteria, fungi, animals and plants) usually contain a composition rich in cationic and hydrophobic amino acids, and having a cationic (positively charged) and amphiphilic characteristics, belonging to the family of cathelicidin and defensin subfamilies (Lei et al; 2019. Am J Transl Res 11:3919-31).

Advantageously, the polycationic peptide antibiotic of the invention is a polymyxin, in particular polymyxin B.

The polymyxins are cationic surfactants that act as antibiotics. Their general structure is that of a polycationic cyclic peptide with a hydrophobic tail. After interaction with the lipid A of lipopolysaccharides, a membrane destabilization occurs leading to cell death. Polymyxins have a bactericidal effect on Gram-negative pathogens, in particular Pseudomonas and Enterobacteriaceae.

In the context of the invention, a “glycopeptide antibiotic” is a glycopeptide antibiotic as defined above that is soluble in water, i.e. having a partition coefficient P of between −3.1 (vancomycin) and 4.10 (oritavancin). The glycopeptide antibiotics comprise a hydrophobic tail composed of 9 or more carbon atoms.

Advantageously, the hydrophobic glycopeptide antibiotic is selected from the group consisting of teicoplanin, vancomycin, daptomycin, telavancin, ramoplanin, decaplanin, corbomycin, complestatin, bleomycin, oritavancin and dalbavancin, in particular teicoplanin.

Advantageously, in the composition of the invention, the antimicrobial peptides are loaded/absorbed onto the microsphere as defined above by non-covalent interactions. This particular way of entrapping drugs or prodrugs is called physical entrapment.

Loading of the antimicrobial peptides onto the microsphere of the invention may be proceeded by numerous methods well-known to the one skilled in the art such as preloading after the microsphere synthesis, or an extemporaneous loading onto preformed and sterilized microspheres.

Advantageously, the composition of the invention comprises between 10 and 300 mg/mL or between 10 and 50 mg/mL of hydrophobic glycopeptide antibiotic, such as teicoplanin, more advantageously between 10 and 30 mg/mL.

Advantageously, the composition of the invention locally releases the hydrophobic glycopeptide antibiotic, such as teicoplanin, at a constant rate of between 100 and 500 μg.

Advantageously, the composition of the invention comprises between 2 and 10 mg/mL of polycationic peptides antibiotic, such as polymyxin B, more advantageously between 2 and 5 mg/mL.

Advantageously, the composition of the invention locally releases the polycationic peptides antibiotic, such as polymyxin B, at a constant rate of between 100 and 500 μg/day.

Advantageously, the composition of the invention releases the antimicrobial peptides so that the local concentration remains over the minimal inhibitory concentration (MIC) for 1 to 30 days, advantageously for 1 to 15 days, preferably for 1 to 7 days, the therapeutic range being between 1 μg/mL to 40 μg/mL.

Composition

In the context of the invention, the composition comprises an effective amount of an antimicrobial peptide, at least one hydrophilic degradable microsphere as defined above, and a pharmaceutically acceptable carrier. The carrier is suitable for administration by injection.

The antimicrobial peptides and the hydrophilic degradable microsphere are as defined above.

According to the invention, the pharmaceutically acceptable carrier is intended for administration of the antimicrobial peptide by injection and is advantageously selected in the group consisting in water for injection, saline, glucose, starch, hydrogel, polyvinylpyrrolidone, polysaccharide, hyaluronic acid ester, contrast agents and plasma.

The composition of the invention can also contain a buffering agent, a preservative, a gelling agent, a surfactant, or mixtures thereof. Advantageously, the pharmaceutically acceptable carrier is saline or water for injection.

The composition of the invention allows the extended-release, in particular the controlled-release, of antimicrobial peptides over a period ranging from a few hours to a few weeks. Advantageously, the composition of the invention allows the controlled-release of antimicrobial peptides for 1 day to 14 days, advantageously for 1 day to 7 days.

The composition of the invention allows the temporal control and the sustained-release as defined above, for example by modulating the nature and the contents of monomers a), b) and/or c).

The invention also relates to the composition as defined above, for use for preventing and/or treating infectious diseases, in particular mammal infectious diseases, by local delivery.

The invention also relates to a method for preventing and/or treating infectious diseases, in particular mammal infectious diseases, comprising locally administering to a subject in need thereof an effective amount of the composition as defined above.

The invention also relates to the use of the composition as defined above for the manufacturing of a drug for preventing and/or treating infectious diseases, in particular mammal infectious diseases, by local delivery. Advantageously, the infectious diseases are caused by Gram-positive bacteria, such as methicillin-resistant Staphylococcus aureus and Enterococcus faecalis, or by Gram-negative bacteria such as Pseudomonas aeruginosa or carbapenemase-producing Enterobacteriaceae.

Advantageously, the infectious diseases caused by methicillin-resistant Staphylococcus aureus are located in skin and soft tissues, but also the bloodstream infections, endocarditis, bone and joint infections, meningitis P. aeruginosa is involved in osteo-articular infections, infections of prosthetic joints, nosocomial infections, or ocular diseases such as conjunctivitis, microbial keratitis, endophthalmitis, blepharitis and dry eye. Gram-positive bacteria are the major contributor of ocular infections (Teweldemedhin et al. 2017. BMC Ophthalmology. 17:212). In keratitis diagnoses, multiple species of bacteria have been identified: P. aeruginosa, E. coli, K. pneumoniae, Acinetobacter, Serratia (S. marcescens and S. liquefaciens), Aeromonas, Fusobacterium, Enterobactor spp., P. mirabilis (Proteus mirabilis), P. multocida (Pasteurella multocida), M. catarrhalis (Moraxella catarrhalis).

The invention also relates to microspheres as disclosed herein for preventing and/or treating virus-related diseases, such as MERS, SARS, and Ebola viruses (Zhou et al; 2016; J Biol Chem. 291: 9218-32) and SARS-CoV-2 (Tripathi et al., 2020; Int J Biol Macromol. 164: 2622-31) based on the anti-viral activity of glycopeptide antibiotics, including teicoplanin, or for the manufacturing of a drug for preventing and/or treating virus-related diseases. Glycopeptides antibiotics, including teicoplanin inhibit at low concentration the cathepsin L protease involved in the cell entry of the virus particles. A sustained delivery of glycopeptide antibiotics from degradable microspheres would achieve a plasma therapeutic concentration to halt the virus replication for patients in early phase of infection which could not be hospitalized. The degradable microspheres loaded with glycopeptides antibiotics could be injected subcutaneously into the deep abdomen for a sustained teicoplanin delivery for several days.

Extemporaneous Loading

In a particular embodiment of the invention, the antimicrobial peptides may be loaded extemporaneously on dry and sterile microsphere.

The invention thus also relates to a pharmaceutical kit comprising:

i) at least one hydrophilic degradable microsphere as defined above in association with a pharmaceutically acceptable carrier for administration by injection;

ii) an effective amount of antimicrobial peptides; and

iii) optionally an injection device,

the hydrophilic degradable microsphere and the antimicrobial peptides being packed separately.

In such an embodiment, antimicrobial peptide is advantageously intended to be loaded on the hydrophilic degradable microsphere just before the injection.

In the context of the invention, “glycopeptide antibiotic” is advantageously a glycopeptide antibiotic as defined above that is soluble in water, i.e. having a partition coefficient P of between −3.1 (vancomycin) and 4.10 (oritavancin). The glycopeptide antibiotic comprises a hydrophobic tail composed of 9 or more carbon atoms.

Advantageously, the hydrophobic glycopeptide antibiotic is selected from the group consisting of teicoplanin, vancomycin daptomycin, telavancin, ramoplanin, decaplanin, corbomycin, complestatin, bleomycin, oritavancin and dalbavancin.

In the context of the invention, a “polycationic peptide” may be a cyclic peptide soluble in water, i.e. having a partition coefficient P between −2.4 (polymyxin E-colistin) and −0.9 (polymyxin B).

The polymyxin scaffold contains a hydrophobic tail, namely a methyl-octanamide chain covalently bound terminal diaminobutyric acid.

In the context of the invention, a “polycationic peptide” may also be a linear peptide soluble in water produced by different organisms (bacteria, fungi, animals and plants) usually contain a composition rich in cationic and hydrophobic amino acids, and having a cationic (positively charged) and amphiphilic characteristics, belonging to the family of cathelicidin and defensin subfamilies (Lei et al; 2019. Am J Transl Res 11:3919-31).

According to the present invention, “injection device” means any device for parenteral administration. Advantageously, the injection device is one or more syringes, which may be pre-filled, and/or one or more catheters or microcatheters.

Use of the microsphere

The invention also relates to the hydrophilic degradable microsphere as defined above for use for the local delivery, advantageously the controlled and local delivery, of an effective amount of antimicrobial peptides to a subject in need thereof.

Advantageously, the sustained delivery of antimicrobial peptides is over a period ranging from a few hours to a few months without burst, advantageously from 1 day to 14 days, more advantageously from 1 day to 7 days.

The invention also relates to a method for locally delivering an effective amount of antimicrobial peptides to a subject in need thereof, advantageously over a period ranging from a few hours to a few months without burst, advantageously from 1 day to 14 days, more advantageously from 1 day to 7 days, comprising the administration of the hydrophilic degradable microsphere as defined above in association with a pharmaceutically acceptable carrier for administration by injection.

The examples which follow illustrate the invention without limiting its scope in any way.

### EXAMPLES

### Example 1: Preparation of Unloaded Microsphere According to the Invention

The starting components and their contents are summarized in Table 1a and 1 b. Table 1a and 1 b also summarizes the main parameters for microspheres synthesis.

TABLE 1a
Formulations of microspheres according to the invention

| | | | Microspheres of 50-100 μm diameter | | | | |
|---|---|---|---|---|---|---|---|
| | Test number | | MS1 | MS2 | MS3 | MS4 | MS5 |
| Process | Oil/Water ratio (V/V) | | 1/11 | 1/11 | 1/11 | 1/11 | 1/11 |
| parameters | Stirring speed | | 240 RPM | 240 RPM | 240 RPM | 240 RPM | 240 RPM |
| | PVA | | 1% | 1% | 1% | 1% | 1% |
| | NaCl | | 3% | 3% | 3% | 3% | 3% |
| Organic | Monomers mass/organic | | 56% | 56% | 56% | 56% | 56% |
| phase | phase mass (wt %) | | | | | | |
| | Toluene (wt %) | | 44% | 44% | 44% | 44% | 44% |
| | Hexanethiol (component d) | | 3% | 3% | 3% | 3% | 3% |
| | (% mole/m-PEGMA mole) | | | | | | |
| | AIBN (% weight/organic | | 0.28% | 0.28% | 0.28% | 0.28% | 0.28% |
| | phase weight | | | | | | |
| | Phase | m-PEGMA | 55% | 51% | 30% | 10% | 55% |
| | monomer | (component a) | | | | | |
| | | (% mole/total | | | | | |
| | | mole monomer) | | | | | |
| | | Crosslinker | 5% of | 9% of | 30% of | 50% of | 5% of |
| | | (component c) | PEG₁₃—PLA₇—PCL₃ | PEG₁₃— | PEG₁₃—PLA₇—PCL₃ | PEG₁₃— | PEG₁₃—PLA₄—PCL₅ |
| | | (% mole/total | | PLA₇—PCL₃ | | PLA₇—PCL₃ | |
| | | mole monomer) | | | | | |
| | | 2-methylene- | 10% | 10% | 10% | 10% | 10% |
| | | 1,3-dioxepane | | | | | |
| | | (MDO) | | | | | |
| | | (component b) | | | | | |
| | | (% mole/total | | | | | |
| | | mole monomer) | | | | | |
| | | Methacrylic acid | 30% | 30% | 30% | 30% | 30% |
| | | (component e) | | | | | |
| | | (% mole/total | | | | | |
| | | mole monomer) | | | | | |

TABLE 1b
Formulations of microspheres according to the invention for antibiotics loading.

| | | | Microspheres of 50-100 μm diameter | | | | | |
|---|---|---|---|---|---|---|---|---|
| | MS number | | MS6 | MS7 | MS8 | MS9 | MS10 | MS11 |
| Process | Oil/Water ratio (V/V) | | 1/11 | 1/11 | 1/11 | 1/11 | 1/11 | 1/11 |
| parameters | Stirring speed | | 240 RPM | 240 RPM | 240 RPM | 240 RPM | 240 RPM | 240 RPM |
| | PVA (%) | | 1 | 1 | 1 | 1 | 1 | 1 |
| | NaCl (%) | | 3 | 3 | 3 | 3 | 3 | 3 |
| Organic | Monomers mass/organic | | 56% | 56% | 56% | 56% | 56% | 56% |
| phase | phase mass (wt %) | | | | | | | |
| | Toluene (wt %) | | 44% | 44% | 44% | 44% | 44% | 44% |
| | Hexanethiol (component d) | | 3% | 3% | 3% | 3% | 3% | 3% |
| | (% mole/m-PEGMA mole or | | | | | | | |
| | tert-butyl methacrylate) | | | | | | | |
| | AIBN (% weight/organic | | 0.28% | 0.28% | 0.28% | 0.28% | 0.28% | 0.28% |
| | phase weight | | | | | | | |
| | Phase | m-PEGMA | 55% | 55% | 85% | 85% | 55% | 0% |
| | monomer | (component a) | | | | | | |
| | | (% mole/total | | | | | | |
| | | mole monomer) | | | | | | |
| | | Tert-butyl | 0% | 0% | 0% | 0% | 0% | 30% |
| | | methacrylate | | | | | | |
| | | (component a) | | | | | | |
| | | (% mole/total | | | | | | |
| | | mole monomer) | | | | | | |
| | | Crosslinker | 5% | 5% | 5% of | 5% | 5% | 30% |
| | | (component c) | PEG₁₃—PLGA₁₂ | PEG₁₃—PLGA₁₂ | PEG₁₃— | PEG 3-arm- | PEG₂—PCL₁₂ | PEG₁₃— |
| | | (% mole/total | | | PLA₇—PCL₃ | PLA₇—PCL₃ * | | PLA₇—PCL₃ |
| | | mole monomer) | | | | | | |
| | | 2-methylene- | 10% | 10% | 10% | 10% | 10% | 10% |
| | | 1,3-dioxepane | | | | | | |
| | | (MDO) | | | | | | |
| | | (component b) | | | | | | |
| | | (% mole/total | | | | | | |
| | | mole monomer) | | | | | | |
| | | Methacrylic acid | 30% | 30% | 0% | 0% | 30% | 30% |
| | | (component e) | | | | | | |
| | | (% mole/total | | | | | | |
| | | mole monomer) | | | | | | |

* the crosslinker is 3 arm PEG with a molar mass of 1014 g/mol, PLA₇—PCL₃ as a total of 10 units.

The aqueous phase solution (917 mL) containing 1 wt % polyvinyl alcohol (Mw=13000-23000 g/mol), 3 wt % NaCl in deionized water was placed in a 1 dm³ reactor and heated up to 50° C.

The organic phase was prepared in an Erlenmeyer. Briefly, toluene (36.9 g) and 2,2′-azobis(2-methylpropionitrile) (AIBN) (0.28 wt %/organic phase weight) were weighted. AIBN was introduced in another vial and solubilized in a volume fraction (≈30%) of the weighted toluene.

Then, degradable crosslinker was weighted in an Erlenmeyer. Polyethylene glycol methyl ether methacrylate (Mn=300 g/mol) or tert-butyl methacrylate (Mn=142.2 g/mol), methacrylic acid and 2-methylene-1,3-dioxepane (MDO) were weighted and introduced into the Erlenmeyer. Then the remaining volume of toluene was added to solubilize the monomers. Hexanethiol (3 mol %/mol of m-PEGMA or tert-butyl methacrylate) was added to the Erlenmeyer using a micropipette. The AIBN solution in toluene was added to the Erlenmeyer containing monomers. Finally, the organic phase had to be clear (monomer and initiator should be totally solubilized) without any aggregates before introduction into the aqueous phase.

The organic phase was poured into the aqueous phase at 50° C. Thereupon, stirring (240 rpm) was applied by using an impeller. After 4 minutes, the temperature was raised up to 80° C. After 8 hours, the stirring was stopped and microspheres were collected by filtration on a 40 μm sieve and washed extensively with acetone and water. Microspheres were then sieved with decreasing size of sieves (125 μm, 100 μm, 50 μm). MS in the size range 50-100 μm were collected for drug loading trials.

### Example 2: Loading of Microspheres According to Example 1 with Teicoplanin or Polymyxin B (Preloading after MS Synthesis)

After the sieving step, 500 μL of microspheres obtained in example 1 (size range 50-100 μm) were placed in 15 mL polypropylene vials. Then, 0.5 up to 2 mL teicoplanin (Sigma T0578-100 mg) or 2 mL of polymyxin B sulfate (Merck ref 5291-500MG) in water at 10 mg/mL were added to the wet microspheres. For the polymyxin B sulfate loading, the solution was completed with 12 mM of sodium bicarbonate. The loading step was performed at room temperature for 1 h under stirring on a tube rotator (≈30 rpm).

Then,
for the teicoplanin loading, the supernatants were removed for the measurement of unbound teicoplanin. Absorbance was measured at 280 nm and the amount of teicoplanin in supernatant was obtained by extrapolation from a standard curve (15 to 500 μg/mL), and the loaded dose was calculated by subtraction;
for the polymyxin B sulfate loading, the supernatants were removed for the measurement of unbound Polymyxin B. Absorbance was measured at 220 nm and the amount of polymyxin B in supernatant was obtained by extrapolation from a standard curve (12.5 to 500 μg/mL), and the loaded dose was calculated by subtraction.

The loaded dose of each peptide was calculated by subtracting the final amount of peptide from the initial amount. The antibiotic loading for 1 mL of beads was obtained by multiplying by 2 the quantity loaded on 0.5 mL of MS. The loading efficiency was calculated by the following equation: Loading efficiency=((Antibiotic in feed−Antibiotic in supernatant)/Antibiotic in feed)×100. The pellets were washed with 10 mL of a solution of glucose in water (2.5% w/v). Then, the microsphere pellets were frozen-dried before e-beam sterilization (15-25 kilograys).

Table 2 summarizes loading for each MS formulation tested.

TABLE 2
Antibiotic loading of the microspheres
according to example 2

| MS | | Antibiotics loading (mg/mL) |
|---|---|---|
| used | Antibiotics | (% of loading efficacy) |
| MS1 | Teicoplanin | 15.6 |
| | | (78%) |
| MS1 | Polymyxin B | 14.3 |
| | | (35%) |
| MS2 | Teicoplanin | 17.5 |
| | | (87%) |

Immediately after the sieving of microspheres of example 1, significant amounts of antibiotics (teicoplanin or polymyxin B) were loaded after 1 h of mixing with MS showing affinity between the peptides and the degradable polymer.

### Example 3: Extemporaneous Loading of Teicoplanin, Dalbavancin, Polymyxin B, Polymyxin E (Colistin) and Human Cathelicidin Peptide LL-37 on Sterile Microspheres According to Example 1

The antibiotic loading reactions were performed on dry and sterile microspheres during a short time period (60 min) by mixing (=30 rpm) at room temperature. Then, the drug release experiments started immediately in PBS (Sigma P-5368; 10 mM phosphate buffered saline; NaCl 0,136 M; KCl 0.0027 M; pH 7.4 at 25° C.) in sink condition at 37° C.

Teicoplanin loading: after the sieving step, a pellet of 0.5 mL of microspheres obtained in example 1 (size range 50-100 μm) in 5 mL of a solution containing 5% (w/v) of glucose was prepared. After homogenization, the supernatant was removed, and the pellet of microspheres was frozen-dried and sterilized by e-beam radiation (15-25 kilograys). Then, teicoplanin (Sigma, T0578) was solubilized in water at 10 or 80 mg/mL before addition of various volumes (1 up to 4 mL) to the dry microsphere pellets. The loading step was done at room temperature for 60 min under stirring on a tube rotator (≈30 rpm). Samples (50 μL) were taken after 15-30-45 and 60 min to analyse the kinetic of teicoplanin loading. After the last sampling, the supernatants were removed. For the measurement of unbound teicoplanin, absorbance of supernatants was measured at 280 nm and the amount of teicoplanin was obtained by extrapolation from a standard curve (15 to 500 μg/mL). The loaded dose of teicoplanin was calculated by subtracting the final amount of peptide from the initial amount. The antibiotic loading for 1 mL of beads was obtained by multiplying by 2 the quantity loaded on 0.5 mL of MS.

Dalbavancin loading: after the sieving step, a pellet of 0.5 mL of microspheres (MS2) obtained in example 1 (size range 50-100 μm) in 5 mL of a solution containing 5% (w/v) of glucose was prepared. After homogenization, the supernatant was removed, and the pellet of microspheres was frozen-dried and sterilized by e-beam radiation (15-25 kilograys). Then, dalbavancin (Sigma, SML2378) was solubilized in water at 5 mg/mL before addition of 2 mL (10 mg) to the dry microsphere pellets. The loading step was done at room temperature for 60 min under stirring on a tube rotator (=30 rpm). Samples (50 μL) were taken after 15-30-45 and 60 min to analyse the kinetic of dalbavanin loading. After 1 h the supernatants were removed. For the measurement of unbound dalbavancin, absorbance of supernatants was measured at 280 nm and the amount of dalbavancin was obtained by extrapolation from a standard curve (15 to 500 μg/mL). The loaded dose of dalbavancin was calculated by subtracting the final amount of peptide from the initial amount. The antibiotic loading for 1 mL of beads was obtained by multiplying by 2 the quantity loaded on 0.5 mL of MS.

Polymyxin B or polymyxin E (colistin) loading: after the sieving step, a pellet of 0.5 mL of microspheres obtained in example 1 (size range 50-100 μm) in 5 mL of a solution containing 5% (w/v) of glucose and 12 mM of sodium bicarbonate was prepared. For MS11, the concentration of sodium bicarbonate was raised to 24 mM. After homogenization, the supernatant was removed, and the pellet of microspheres was frozen-dried and sterilized by e-beam radiation (15-25 kilograys). Then, 1 mL of polymyxin B (Merck, 5291-500MG) or polymyxin E (Sigma, C4401) in solution at 10 mg/mL in water was added to the sterile and dry microspheres. The loading step was done at room temperature for 15 min under stirring on a tube rotator (˜ 30 rpm). Samples (20 μL) were taken at 5-10 and 15 min to analyse the kinetic of polymyxin loading. Then, after 15 min, the supernatants were removed. Absorbance of supernatants was measured at 220 nm and the amount of polymyxin B or polymyxin E was obtained by extrapolation from a standard curve (12.5 to 500 μg/mL). The loaded doses of polymyxins were calculated by subtracting the final amount of peptides from the initial amounts. The antibiotic loading for 1 mL of beads was obtained by multiplying by 2 the quantity loaded on 0.5 mL of MS.

Peptide LL-37 (human cathelicidin peptide) loading: after the sieving step, pellets of 100 μL of microspheres (MS2) obtained in example 1 (size range 50-100 μm) in 5 mL of a solution containing 5% (w/v) of glucose and 12 mM of sodium bicarbonate were prepared. After homogenization, the supernatant was removed, and the pellets of MS2 were frozen-dried and sterilized by e-beam radiation (25 kilograys). Then, 0.5 mL of the antimicrobial peptide LL-37 with the sequence LLGDFFRKSKEKIGKEFKRIVQRIKDFLRNLVPRTES (Theoretical pl/Mw: 10.61/4493.32) (SB-peptide, Saint Egrbve, France) in solution at 2 mg/mL in water was added to the sterile and dry microspheres. The loading of peptide on MS2 was done at room temperature under stirring on a tube rotator (˜ 30 rpm). Samples (20 μL) were taken at 5-10 and 15 min to analyse the kinetic of peptide loading. The measurement of unbound peptide was done by RP-HPLC on a C₄ column (ACE 5 C4, 150×4.6 mm) with detection at 210 nm. The mobile phase used in chromatographic separations consisted of a binary mixture of 2 solvents: water with 0.1% TFA (A) and acetonitrile with 0.1% TFA (B) at a flow rate of 1 mL/min at 25° C. Solvent B raised from 40% to 85% in 6 min before 4 min of equilibration in 60% of solvent A. The amount of unbound peptide LL-37 was obtained by extrapolation from a standard curve (0.5 to 50 μg/mL). The loaded dose of LL-37 peptide was calculated by subtracting the final amount of peptide from the initial amount. The LL-37 peptide loading for 1 mL of beads was obtained by multiplying by 10 the quantity loaded on 100 μL of MS2.

For each antibiotic, the loading efficiency was calculated by the following equation: loading efficiency=((Antibiotic in feed−Antibiotic in supernantant)/Antibiotic in feed)×100.

Table 3 summarizes the results of the extemporaneous loading experiments done with glycopeptides including teicoplanin and dalbavancin for the different formulations.

TABLE 3
Time course loading of glycopeptide antibiotics
(teicoplanin and dalbavancin) on degradable microspheres according to example 3.

| | | Antibiotics loading (mg/mL) | | | | Log P value |
|---|---|---|---|---|---|---|
| | | (% of loading efficiency) | | | | of degradable |
| Antibiotics | MS used | 15 min | 30 min | 45 min | 60 min | crosslinkers |
| Teicoplanin | MS1 | 38.2 ± 7.1 | ND | ND | ND | 3.2 |
| | | (88%) | | | | |
| | MS2 | 47.8 ± 20.1 | 69 ± 0.8 | ND | ND | 3.2 |
| | | (75%) | (86%) | | | |
| | MS3 | 18.1 ± 4.8 | 22.4 ± 0.4 | 25.5 ± 7.6 | 26.9 ± 8.2 | 3.2 |
| | | (51%) | (63%) | (71%) | (74%) | |
| | MS4 | 12.2 ± 2.7 | 15.9 ± 3.5 | 19.6 ± 4.1 | 22.6 ± 3.1 | 3.2 |
| | | (29%) | (38%) | (46%) | (54%) | |
| | KW* | p = 0.0002 | p = 0.0023 | NA | NA | — |
| | MS5 | 36 | ND | ND | ND | 4.1 |
| | | (90%) | | | | |
| | MS8 | 32.8 ± 1.9 | 38.2 ± 0.3 | ND | MD | 3.2 |
| | | (65%) | (73%) | | | |
| | MS9 | 33.1 ± 2.3 | 37 ± 0.7 | ND | ND | 3.2 |
| | | (66%) | (74%) | | | |
| Dalbavancin | MS2 | 5.8 ± 1.1 | 9.8 ± 0.4 | 9.8 ± 0.5 | 9.8 ± 0.2 | 3.2 |
| | | (29%) | (49%) | (49%) | (49%) | |

*KW: non-parametric kurskall-Wallis test to compare the effect of crosslinker content on teicoplanin efficiency loading for MS1, MS2, MS3 and MS4. The significance was set at p < 0.05.
NA: not-applicable.
ND: not determined.

Extemporaneous loading of teicoplanin on dry and sterile MS occurred rapidly in water at room temperature with variable yields (Table 3). Teicoplanin is loadable on degradable with or without methacrylic acid monomer with good efficacy. For the MS containing 30% of methacrylic acid (MS1 to MS4), the crosslinker content had a significant effect on efficiency of teicoplanin loading. After 15 minutes of mixing with the antibiotic, the yield of loading decrease with the content of crosslinker: 88% for 5 mol % of crosslinker, 75% for 9 mol % of crosslinker, 51% for 30 mol % of crosslinker and 29% for 50 mol % of crosslinker. For the highly crosslinked microspheres (MS3 and MS4), the loading reaction was slow, the efficiency of loading increased slowly during the time to reach after 1 h of mixing a yield of 74-54% for MS3 and MS4, respectively.

The loading efficiency of dalbavancin on MS2 reached a plateau after 30 min, with a low loading efficiency of ˜50%. This difference of loading yield could not be explained by the molecular weight of the glycopeptides (teicoplanin: 1879.7 g/mol and dalbavancin: 1816.7 g/mol) which are very close.

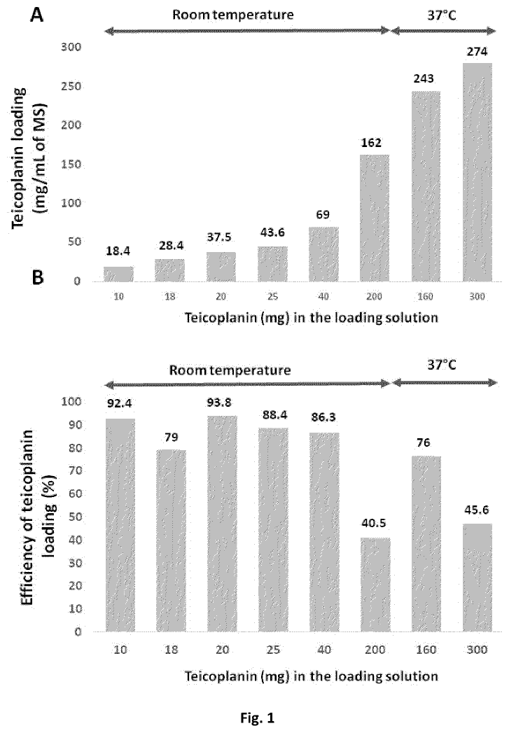

The effect of the amount of teicoplanin in the feed solution on the drug loading was analysed on MS2 (FIG. 1).

The teicoplanin loading on degradable MS of example 1 was tuneable according to the amount of drug present in the feeding solution. High amount of glycopeptide (160 mg/mL) was loaded on degradable microspheres showing a strong affinity between teicoplanin and the degradable polymer. The amount of teicoplanin loaded on MS2 was improved when the loading reactions were performed at 37 C, a payload close to 300 mg of antibiotic was achieved for 1 mL of beads.

The results of the extemporaneous loading experiment of cationic peptides including polymyxin B, polymyxin E (colistin) and human cathelicidin peptide LL-37 for each MS formulation tested are summarized in Table 4.

TABLE 4
Summary of the time course loading of cationic antibiotic peptides (polymyxin B,
polymyxin E (colistin) and human cathelicidin peptide LL-37) on degradable
microspheres according to example 3. The partition coefficient P (Log P) values
of degradable crosslinkers were determined in silico.

| | | | | | Log P value |
|---|---|---|---|---|---|
| | | Antibiotics loading (mg/mL) | | | of |
| | | (% of loading efficiency) | | | degradable |
| Antibiotics | MS used | 5 min | 10 min | 15 min | crosslinkers |
| Polymyxin | MS1 | 8.2 ± 3.2 | 9.1 ± 1.9 | 9.5 ± 1.9 | 3.2 |
| B | | (41%) | (45%) | (47%) | |
| | MS2 | 14.6 ± 0.4 | 14.6 ± 0.4 | 14.7 ± 0.5 | 3.2 |
| | | (73%) | (73%) | (73%) | |
| | MS5 | 10.6 ± 4.7 | 10.6 ± 4.7 | 10.7 ± 4.8 | 4.1 |
| | | (53%) | (53%) | (53%) | |
| | MS6 | 13.6 ± 0.2 | 12.4 ± 1.2 | 12.8 ± 1 | ND |
| | | (68%) | (62%) | (64%) | |
| | MS7 | 13.3 ± 0.8 | 13.3 ± 0.5 | 13 ± 0.8 | 0.5 |
| | | (66%) | (66%) | (65%) | |
| | MS8 | 0.25 ± 0.03 | 0 | 0 | 3.2 |
| | | (1.3%) | | | |
| | MS10 | 13.2 ± 0.2 | 12.7 ± 0.7 | 12.9 ± 0.1 | 11.2 |
| | | (68%) | (62%) | (64%) | |
| | MS11 | 12.5 ± 0.1 | 14.6 ± 0.1 | 15.1 ± 0.2 | 3.2 |
| | | (62%) | (73%) | (75%) | |
| Polymyxin | MS2 | 13.4 ± 0.49 | 13.1 ± 0.4 | 13.1 ± 0.63 | 3.2 |
| E | | (67%) | (65%) | (65%) | |
| | MS5 | 14.1 ± 1.45 | 13.9 ± 1.09 | 14.4 ± 1.22 | 4.1 |
| | | (70%) | (69%) | (72%) | |
| Peptide | MS2 | 9.9 ± 0.019 | 9.9 ± 0.029 | 9.9 ± 0.019 | 3.2 |
| LL-37 | | (99.5%) | (99.7%) | (99.7%) | |

The loading of polymyxin B occurred on anionic MS containing 30 mot % of methacrylic acid monomer showing the electrostatic interaction with the cationic cyclopeptide. No loading was observed on MS8 which is devoid of negative charges. On the sterile anionic MS of example 1, the extemporaneous loading of polymyxin B was fast, a plateau was reached after 5 min of mixing. The loading efficiency was between 50% and 75%. Longer incubation time did not improve the loading yields. When the hydrophilic monomer m-PEGMA was replaced by another main monomer, the tert-butyl methacrylate (MS11), the extemporaneous loading of polymyxin B occurred with an efficiency similar to other microspheres.

The loading of polymyxin E, a variant of polymxyin B (replacement of a phenylalanine with a leucine residue) was also feasible on degradable MS of example 1 at 5 mol % or 9 mol % of crosslinker (Table 4). Polymyxins of different compositions are loadable in few minutes on dry and sterile microspheres of example 1 according to a simple loading method.

The loading of the linear cationic peptide LL-37 was fast and efficient on MS2; a loading of ˜10 mg of peptide was achieved after 5 min of contact with MS2 with a yield close to 100%. Loading of higher amount of human cathelicidin peptide was predictable on degradable MS of example 1 containing methacrylic acid.

### Example 4. Study of the In Vitro Release of Glycopeptide Antibiotics Including Teicoplanin and Dalbavancin and Cationic Antibiotic Peptides, Including Polymyxin B, Polymyxin E (Colistin) and Peptide LL-37 from Microspheres Loaded According to Example 3

After the removal of the different loading solutions, the elution of antibiotics loaded extemporaneously on dry and sterile MS was done in PBS (Sigma, P-5368; 10 mM phosphate buffered saline; NaCl 0,136 M; KCl 0.0027 M; pH 7.4) at 37° C. under shaking (150 rpm), the tubes were placed horizontally in the oven. At each sampling time, the medium was completely renewed with fresh PBS (1 mL for human cathelicidin peptide LL-37; 14 mL for teicoplanin and polymyxins and 40 mL for dalbavancin).

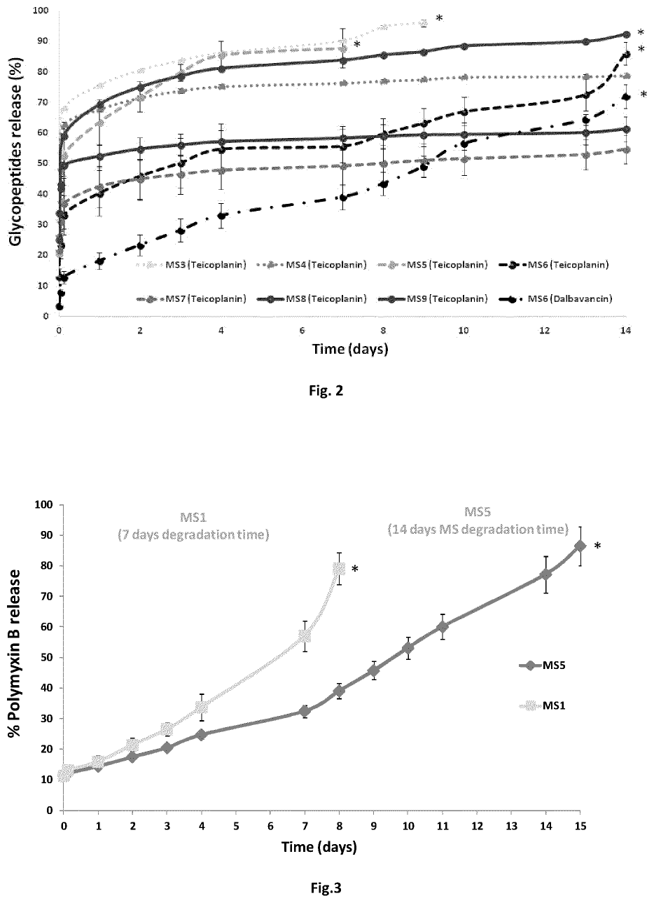

The amounts of teicoplanin and dalbavancin released in PBS supernatants were determined by UV spectrometry at 280 nm by extrapolation from standard curves (15 to 500 μg/mL for teicoplanin and 1 to 50 μg/mL for dalbavancin) (Table 5 and FIG. 2). The assay of polymyxins (B and E) eluted in PBS was done by RP-HPLC on a C₁₈ column (ACE, Super C18, 150×4.6 mm) with detection at 205 nm (Table 6 and FIG. 3). The mobile phase used in chromatographic separations consisted of a binary mixture of 2 solvents: water with 0.075% TFA (A) and acetonitrile with 0.075% TFA (B) at a flow rate of 1 mL/min at 25° C. Solvent B raised from 10% to 90% in 10 min before 4 min of equilibration in 90% of solvent A. The amount of polymyxins in PBS was obtained by extrapolation from a standard curve (10 to 400 μg/mL). The assay of peptide LL-37 eluted in PBS was done by RP-HPLC on a C₄ column (ACE 5 C4, 150×4.6 mm) with detection at 210 nm, solvent B (acetonitrile with 0.1% TFA) raised from 40% to 85% in 6 min before 4 min of equilibration in 60% of solvent A (water with 0.1% TFA). The amount of released peptide LL-37 was obtained by extrapolation from a standard curve (0.5 to 50 μg/mL). The degradation products of MS2 during the release of peptide LL-37 in PBS was quantified according to a colorimetric assay specific of pegylated compounds (Gong et al., 2007. Talanta, 71:381-4).

The table 5 summarizes the in vitro release values of glycopeptides (teicoplanin and dalbavancin) for each MS formulation tested.

TABLE 5
In vitro analysis of glycopeptide antibiotics release done in PBS (37° C., 150 rpm)
immediately after the extemporaneous drug loading on the dry and sterile microspheres.

| | | % of eluted glycopeptides in PBS | | | | | |
|---|---|---|---|---|---|---|---|
| Antibiotics | MS used | 10 min | 1 h | 3 h | J1 | J7 | J14 |
| Teicoplanin | MS1 | 20.6 ± 6 | 37.7 ± 9.4 | 52.4 ± 9.5 | 63.5 ± 7.4 | 87.6 ± 6.4 | NA |
| | MS2 | 13 ± 2.5 | 23.4 ± 4.4 | 33.1 ± 6.3 | 40.3 ± 7.3 | 55.7 ± 5.4 | 85.9 ± 3.7 |
| | MS3 | 21.7 ± 7.4 | 31.1 ± 7.4 | 36.9 ± 8.2 | 42.4 ± 6.7 | 49.2 ± 6 | 54.6 ± 4.7 |
| | MS4 | 33.8 ± 2.3 | 43.1 ± 3.4 | 49.4 ± 3.4 | 52.4 ± 3.6 | 58.3 ± 3.8 | 61.3 ± 4.1 |
| | MS5 | 25 | 41.7 | 58.9 | 69.2 | 83.7 | 92.2 |
| | MS8 | 31.6 ± 0.3 | 53.7 ± 0.1 | 67.9 ± 0.1 | 75.5 ± 0.1 | 90 ± 0.2 | 95.9 ± 1.1 |
| | MS9 | 26.7 ± 0.3 | 48.2 ± 2.2 | 62.4 ± 1.2 | 67.6 ± 0.8 | 76.2 ± 0.5 | 78.7 ± 0.8 |
| | KW* | p = 0.1368 | p = 0.1368 | p = 0.1368 | p = 0.1986 | p = 0.2539 | NA |
| Dalbavancin | MS2 | 3.4 ± 0.8 | 7.8 ± 1 | 12.8 ± 2 | 18.2 ± 2.7 | 39 ± 4 | 71.9 ± 3.9 |

*time of degradation of microspheres in PBS during the antibiotics release.
KW*: non-parametric kurskall-Wallis test to analyse the teicoplanin release for MS at 5 mol % crosslinker (MS1, MS5, MS8, MS9). The significance was set at p < 0.05.
NA: not-applicable (degraded MS).

The in vitro elution of teicoplanin depends on the microsphere composition (Table 5). An important drug release occurred during the washing of loaded MS in PBS during 10 min, 13 to 33% of the payload according to the MS. Then, the teicoplanin release raised during the time, up to microspheres degradation for MS1, MS2, MS5 and MS8. For the MS at 5 mol % of crosslinker (MS1, MS5, MS8, MS9) the effect of methacrylic acid on teicoplanin release was not significant. Methacrylic acid speeds up the MS degradation, 1 week for MS1 and 2 weeks for MS8 which both were at 5 mol % of crosslinker PEG₁₃-PLA₇-PCL₃.

During the first days in PBS, the release values were lower for MS2 at 9 mol % of crosslinker compared to MS at 5 mol %, 30 mol % or 50 mol % of crosslinker content. For the highly crosslinked microspheres (MS3 and MS4) which were not degraded during the experiment, an important burst release occurred at 10 min and at 1 h followed by a slight drug elution during 2 weeks. These highly crosslinked MS (30 mol % and 50 mol % of crosslinker) are not efficient carriers for teicoplanin since the drug loading was slow (Table 4) and the early release reaction was fast. A high hydrogel crosslinking hindered an efficient controlled and sustained delivery, probably because a part of the teicoplanin molecules are weakly bound to the material due to the small mesh size of the hydrogel which probably maintains teicoplanin at the microsphere surface (molecules removed during the burst), while another part of the antibiotic molecules remain probably firmly entrapped within the polymer network and were eluted at a low flow rate during the time.

There is an optimum degree of hydrogel crosslinking (≈9 mol %) which allowed both efficient loading and good diffusion of the molecules through the polymer during MS degradation. Introduction of a more hydrophobic crosslinker in MS5 (PEG₁₃-PLA₄-PCL₅) compared to PEG₁₃-PLA₇-PCL₃ (MS1) did not change the release profile of teicoplanin during the first week, showing the importance of MS crosslinking rather than the crosslinker hydrophobicity for the drug release control.

The elution of dalbavancin from MS2 occurred in a more sustained way than for teicoplanin, probably because of the more hydrophobic nature of dalbavancin whose fatty acid side chain contains one more carbon group than teicoplanin which probably increase hydrophobic interaction with the crosslinker.

The duration of glycopeptides elution depends on the crosslinker composition and content in the degradable MS (FIG. 2).

MS at 5 mol % of crosslinker PEG₁₃-PLA₇-PCL₃ (MS1) was degraded at 1 week while at 9 mol % of crosslinker the degradation occurred after 2 weeks (MS2). At 30 mol % and 50 mol % of crosslinker, the MS were not degraded after 2 weeks in PBS. MS5 at 5 mol % of crosslinker PEG₁₃-PLA₄-PCL₅ was degraded after 2 weeks but the initial burst was important. The glycopeptides release (teicoplanin, dalbavancin) followed the degradation of microspheres. MS2 at 9 mol % of crosslinker allowed a sustained release of both glycopeptides during 2 weeks with a low burst (3.4% for dalbavancin and 13% for teicoplanin after 10 minutes in PBS).

The table 6 summarizes the in vitro release values of cationic peptides (polymyxin B and polymyxin E and peptide LL-37) for each MS formulation tested.

TABLE 6
In vitro analysis of polymyxins release done in PBS (37° C., 150 rpm)
immediately after the extemporaneous loading on dry and sterile microspheres.

| | | % of eluted polymyxins in PBS | | | | | |
|---|---|---|---|---|---|---|---|
| Antibiotics | MS used | 10 min | 3 h | J1 | J4 | J7 | J14 |
| Polymyxin B | MS1 | 11.3 ± 1.8 | 13.1 ± 1.7 | 15.9 ± 1.9 | 33.7 ± 4.2 | 57 ± 5 | NA |
| | MS2 | 1.6 ± 0.1 | 1.6 ± 0.1 | 1.7 ± 0.1 | 3.1 ± 0.3 | 6.8 ± 0.7 | 50.8 ± 3.8 |
| | MS5 | 11.3 | 12.1 | 14.9 ± 0.1 | 24.6 ± 1.1 | 32.4 ± 1.9 | 77.2 ± 5.9 |
| | MS6 | 31.5 ± 0.8 | 66.7 ± 1.9 | 112.2 ± 2 | NA | NA | NA |
| | MS7 | 7.4 ± 0.4 | 9.6 ± 0.4 | 22.8 ± 0.8 | 74.5 ± 2.2 | NA | NA |
| | MS10 | 1.7 ± 0.2 | 1.7 ± 0.2 | 1.7 ± 0.2 | 2.1 ± 0.4 | 2.1 ± 0.4 | 2.5 ± 0.4 |
| | MS11 | 0.5 ± 0.1 | 0.6 ± 0.2 | 0.8 ± 0.2 | 1 ± 0.2 | 1.1 ± 0.2 | 1.3 ± 0.2 |
| Polymyxin E | MS2 | 6.9 ± 0.9 | 7.7 ± 3.1 | 8.6 ± 1.1 | 13.4 ± 2.3 | 19.5 ± 3.2 | 57.2 ± 8.9 |
| | MS5 | 13.2 ± 2.5 | 17.6 ± 3.1 | 21.2 ± 3.7 | 33.5 ± 5.5 | 42.1 ± 6.4 | 73.1 ± 7.8 |
| | MW* | p = 0.0105 | p = 0.0105 | p = 0.0105 | p = 0.0105 | p = 0.0105 | p = 0.0105 |

MW*: non-parametric Mann-Whitney test to compare the effect of MS composition on polymyxin E release. The significance was set at p < 0.05.
NA: not-applicable (degraded MS).

Immediately after the addition of PBS to the antibiotic loaded MS, different amounts of polymyxin B were eluted from degradable MS, from 0.5% for MS11 up to 31% for MS6, showing a low affinity of polymyxin B for MS at 5 mol % of the PEG₁₃-PLGA₁₂ crosslinker. Then, the polymyxins elution in PBS raised with the time up to the MS degradation for MS1, MS2, MS5, MS6 and MS7 (Table 6 and FIGS. 3 & 4). The slowly degradable microspheres, MS10 (5 mol % of hydrophobic PEG₂-CL₁₂ crosslinker) and MS11 (30 mol % of crosslinker and 30 mol % of the hydrophobic tert-butyl methacrylate monomer) released small amounts of polymyxin B during the first 2 weeks of incubation in PBS, showing the correlation between MS degradation and antibiotic elution.

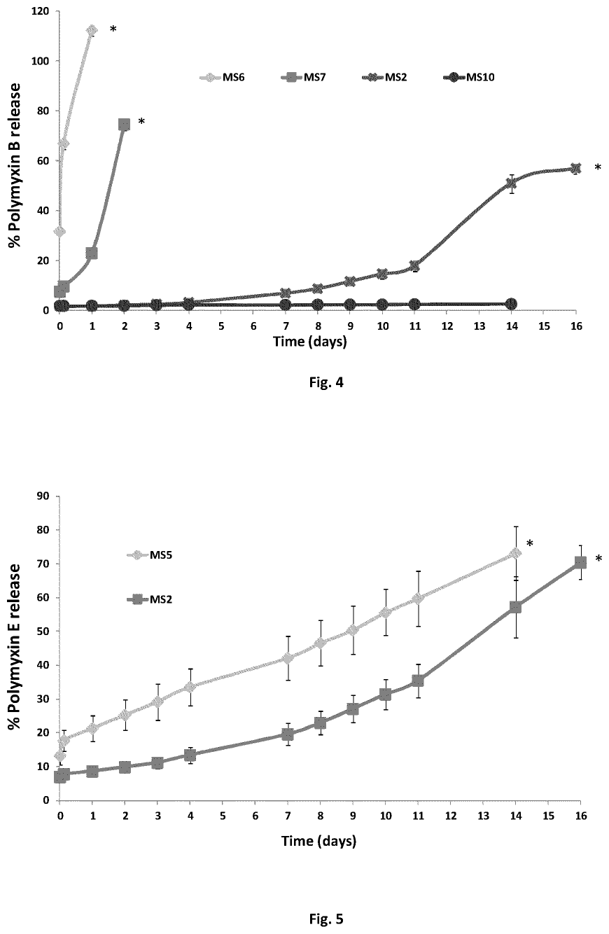

The release of polymyxin B (FIG. 4) and polymyxin E (FIG. 5) from MS2 at 9 mol % of the crosslinker PEG₁₃-PLA₇-PCL₃ was low during the first week of incubation in PBS probably by an important entrapment of cationic peptides within the polymer network, where steric hindrance and electrostatic interactions between the positively charged peptides and negative charges generated during crosslinker hydrolysis probably coexist. At 9 mol % of crosslinker, a burst of polymyxins occurred at the end of MS degradation.

A more sustained release of polymyxins B and E was achieved with MS at 5 mol % of crosslinker (MS1 and MS5), where the initial burst was low and the release was more sustained up to MS degradation. The degree of crosslinking of the hydrogel controls the release reaction of the cationic polymyxin peptides from the degradable microspheres of example 1.

The sustained release of polymyxin B and polymyxin E was tunable according to the degradation time of the microspheres: 1 day for MS6, 2 days for MS7, 1 week for MS1, 2 weeks for MS2 and MS5 and nearly no elution for MS10 which degrade slowly in PBS (FIG. 3, FIG. 4, FIG. 5). For the same crosslinker composition, a microsphere at 5 mol % (MS1) was degraded in one week and in two weeks at 9 mol % of crosslinker (MS2). When the hydrophobicity of the crosslinker increased (MS1 vs MS5) the degradation time of microspheres increased from 1 week to 2 weeks (FIG. 3). The duration of polymyxins B and E release from degradable MS depends on the crosslinker composition and content.

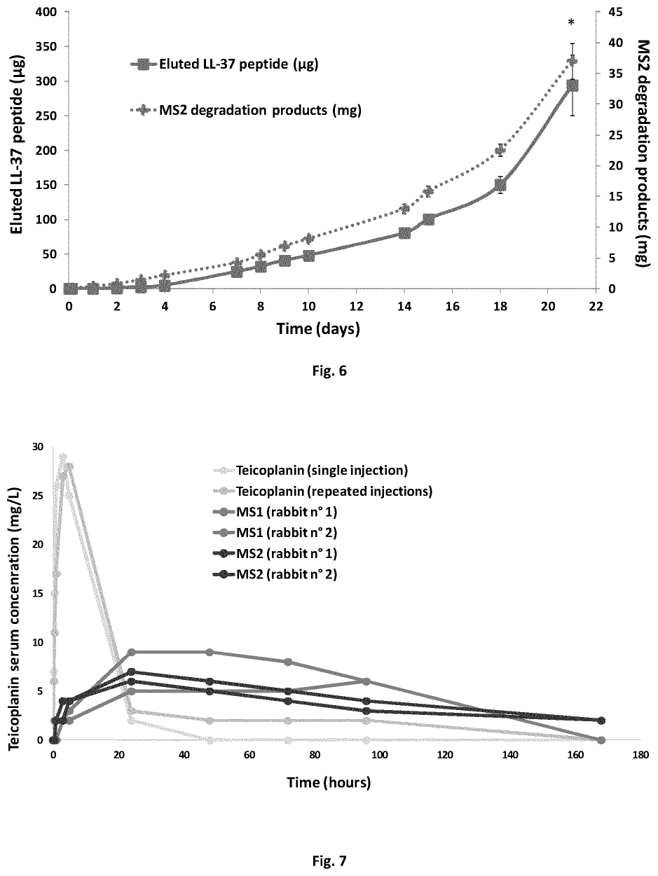

For the human cathelicidin peptide LL-37, the elution in PBS after the extemporaneous loading on MS2 occurred without a burst. The elution progressively increased during the time up to microspheres degradation after 21 days. A strong positive correlation exists between the release of LL-37 peptide and the advancement of MS2 degradation (rho=0.986, p=0.0004) (FIG. 6).

The strong interaction between the 37 amino-acids peptide and the degradable polymer could be explained by electrostatic interactions between the numerous positively charged lysine (16%) and arginine (13%) amino-acid residues of the peptide and the carboxylate functions of the degradable MS2, and probably through hydrophobic interaction established between polymer and the hydrophobic amino-acids (leucine (10%), phenylalanine (10%), valine (5%)) of LL-37 peptide. A sustained delivery of polycationic and amphiphilic peptides belonging to the family of cathelicidin and defensin subfamilies could be achieved with degradable MS of example 1 containing methacrylic acid.

### Example 5: Study of the Plasma Teicoplanin Concentration During the Time Following Single Subcutaneous Injection in Rabbit of Degradable Microspheres Loaded with Teicoplanin According to Example 3

The study was performed in 6 White New Zealand male rabbits (body weight: 3.63±0.17 kg; min-max 3.47-3.88 kg) and included MS1 and MS2 loaded at 40 mg/mL of teicoplanin (Sigma, T-0578).

Briefly, teicoplanin was reconstituted in water for injection at the concentration of 10 mg/mL (100 mL). Sterile dry beads (≈6 mL in 5% glucose) were hydrated/loaded in the teicoplanin solution (20 mL) with continuous agitation on rotating wheel (≈30 rpm). After 20 min, the supernatant was retrieved and kept for drug quantification. Microspheres (MS1 and MS2) were then washed in water, and kept on shell until the time of administration. At the time of injection, MS were suspended in saline and the appropriate volume of bead (≈4 mL) suspension was sampled for injection at a ratio of 2:1 MS/saline (Table 7). Each microsphere was administered once subcutaneously at the same dose of drug at two injection sites to 2 animals. Teicoplanin control (Sigma, T-0578) was administered once in one animal and repeated daily in another animal. Injections were performed subcutaneously in the back of the animal at two different injection sites with a 10 mL Luer syringe and a 18-21 G needle at the total dose.

The dose administered to each rabbit was chosen based on loading and maintenance recommended doses in human, respectively 3 injections of 400 mg followed by 6 mg/kg injections. Additionally, therapeutic levels were achieved in rabbits with experimental bacterial infection with a single dose of 18 mg/kg, which was used as a loading dose. For the maintenance dose, it was estimated that microspheres may release their drug content on a period of 5 days, corresponding to a total maintenance dose of 4 days×6 mg/kg, i.e. 24 mg/kg (Table 7).

TABLE 7
Summary of the animal groups and the teicoplanin doses used for the
subcutaneous injections for the pharmacokinetic study in rabbit
*corresponding to 18 mg/kg at D1, then 6 mg/kg from D2 to D5,
**corresponding to same cumulative dose as animal #2

| Animal # | Article | Injection | Dose | Volume of article (mL) |
|---|---|---|---|---|
| 1 | Injectable | Single | 18 mg/kg | 6.2 |
| | teicoplanin | | | |
| 2 | Injectable | Repeated | 42 mg/kg* | 6.8 + 2.3 |
| | teicoplanin | | | (day 1 up to day 5) |
| 3 | MS1 | Single | 42 mg/kg** | 4.4 |
| 4 | MS1 | Single | 42 mg/kg** | 4.1 |
| 5 | MS2 | Single | 42 mg/kg** | 4.0 |
| 6 | MS2 | Single | 42 mg/kg** | 4.0 |

Animals receiving teicoplanin loaded MS were anesthetized by intramuscular injection of 10 mg/kg ketamine and 2.5 mg/kg xylasine and maintained with oxygen (100%) and Isoflurane (1 to 3%) delivered through a mask. Monitoring included peripheral arterial oxygen saturation (SPO₂) and heart rate. Analgesic treatment with 30 μg/kg buprenorphine was administered before article administration. Animals receiving control teicoplanin were not anesthetized and did not receive analgesic treatment at the time of administration.

A vascular access was gained in the auricular artery for blood sampling and in auricular vein for perfusion. Blood samples (−1.2 mL) were taken on 4 mL dry tubes through an auricular artery to assess the systemic concentration of teicoplanin at the following time points: pre-injection (TO), 15 min, 30 min, 1 h, 3 h, 5 h, 24 h, 2 days, 3 days, 4 days and 7 days. After 1-2 hours, samples were centrifuged at 1800 g for 12 m. to obtain serum (>200 μL) for storage at −80° C. until assay. Assay of teicoplanin in serum samples was performed by Particle Enhanced Turbidimetric Immuno Assay (PETIA) on a Cobas 6000 (Roche Diagnostics, Basel, Switzerland) with QMSO Teicoplanin (Thermo Fisher Scientific, Waltham Mass., USA, Ref. 0374645) as IVD reactive. The lowest limit of quantification of the method (LOQ) is 2.0 mg/L. The results of plasmatic assay of teicoplanin are reported in table 8 and in FIG. 7.

TABLE 8
Individual serum teicoplanin concentration at each time point for each animal.

| | | Cumulative | | | | | | | | | | | |
|---|---|---|---|---|---|---|---|---|---|---|---|---|---|
| Animal | | dose (mg) of | Teicoplanin serum concentration (mg/L) | | | | | | | | | | |
| # | Article | teicoplanin | T0 | 15 min | 30 min | 1 h | 3 h | 5 h | 24 h | 48 h | 72 h | 9 h | 7 days |
| 1 | Injectable | 62.4 | <2.0 | 7.0 | 15.0 | 26.0 | 29.0 | 25.0 | 2.0 | <2.0 | <2.0 | <2.0 | <2.0 |
| | teicoplanin | | | | | | | | | | | | |
| | (single) | | | | | | | | | | | | |
| 2 | Injectable | 160.5 | <2.0 | 6.0 | 11.0 | 17.0 | 27.0 | 28.0 | 3.0 | 2.0 | 2.0 | 2.0 | <2.0 |
| | teicoplanin | | | | | | | | | | | | |
| | (repeated) | | | | | | | | | | | | |
| 3 | MS1 | 162.8 | <2.0 | <2.0 | 2.0 | 2.0 | 2.0 | 2.0 | 5.0 | 5.0 | 5.0 | 6.0 | <2.0 |
| 4 | MS1 | 152.8 | <2.0 | <2.0 | <2.0 | <2.0 | 2.0 | 3.0 | 9.0 | 9.0 | 8.0 | 6.0 | <2.0 |
| 5 | MS2 | 148.0 | <2.0 | <2.0 | <2.0 | 2.0 | 2.0 | 4.0 | 7.0 | 6.0 | 5.0 | 4.0 | 2.0 |
| 6 | MS2 | 146.2 | <2.0 | <2.0 | 2.0 | 2.0 | 4.0 | 4.0 | 6.0 | 5.0 | 4.0 | 3.0 | 2.0 |

Bold figure shows Cₘₐₓ in the serum samples.

Table 8 and FIG. 7 shows individual serum teicoplanin concentration at each time point for each animal. Serum drug concentration profiles were different between injectable teicoplanin and degradable MS1 t MS2 and similar between the two MS.

Time-concentration curves for control animals showed a peak serum concentration of 29.0 and 28.0 mg/L at 3 hours and 5 hours, respectively. Serum level of drug then decreased to 2.0 and 3.0 mg/L at 24 h. For the animal receiving a single dose of drug, teicoplanin concentration was below the LOQ of 2.0 mg/L at 48 h and after. For the animal receiving repeated dosage of teicoplanin, concentration was maintained above 2.0 mg/mL for all the period of drug administration and decreased below LOQ at 7 days (3 days after the last dosage).

For the 2 microspheres batches, teicoplanin serum concentration increased progressively after administration to a Cₘₐₓ between 6.0 and 9.0 mg/L that was generally reached after 24 h. These drug levels were maintained for approximately 4 days and decreased after 7 days. One week after the MS implantation, serum teicoplanin was below LOQ for the two animals receiving the MS1 with a shorter time of degradation (1 week in PBS) while they were still above LOQ for the two animals receiving MS2 (2 weeks in PBS) with a longer time of degradation (FIG. 2).

A flash extemporaneous loading of teicoplanin on dry and sterile degradable microspheres allows a controlled and sustained delivery of the antibiotic in vivo without a burst, the duration of drug release depends on the microsphere degradation time. This in vivo test confirms the strong affinity existing between teicoplanin and degradable microspheres composed of polyethylene glycol. The degradable microspheres of example 1 are efficient platforms of teicoplanin for a sustained in vivo delivery.

### Example 6: Size Distribution of Hydrophilic Microspheres (MS1 and MS2) after Synthesis

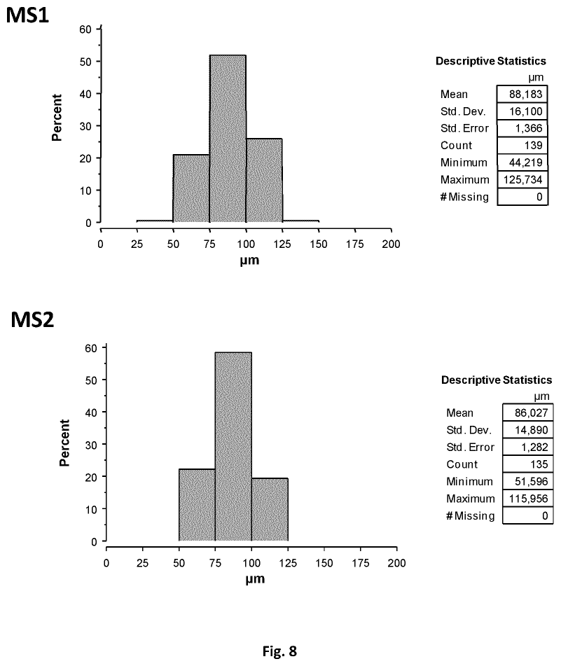

Degradable microspheres used for the pharmacokinetic study have similar size distribution after the sieving step (50-100 μm) as described in example 1 (FIG. 8).

1. A composition comprising an effective amount of a peptide antibiotic, at least one hydrophilic degradable microsphere comprising a crosslinked matrix, and a pharmaceutically acceptable carrier for administration by injection, the crosslinked matrix being based on at least:
a) from 10 mol % to 90 mol % of a hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, —(CH₂—CH₂—O)ₘ CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 mol % to 30 mol % of a cyclic monomer of formula (II): 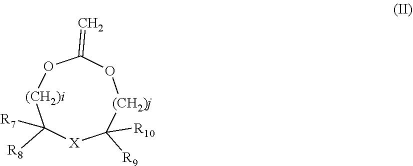
wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;
and
c) from 5 mol % to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:
(CH₂═CR₁₁)—CO—Xₙ-PEGₚ-Xₖ—CO—(CR₁₁═CH₂) (IIIa); or
W(PEGₚ-Xₙ—O—CO—(CR₁₁═CH₂))_{z} (IIIc);
wherein
R₁₁ is independently a hydrogen atom or a (C₁-C₆)alkyl group;
Xₙ or Xₖ is independently PLA, PGA, PLGA, PCL or PLAPCL;
n and k are independently integers from 1 to 150;
W is a carbon atom, a C₁-C₆-alkyl group or an ether group comprising 1 to 6 carbon atoms;
p is an integer from 1 to 100;
z is an integer from 3 to 8;
wherein mol % of components a) to c) are expressed relative to the total number of moles of compounds a), b) and c).

2. The composition of claim 1, wherein the degradable block copolymer cross-linker c) is selected from the group consisting of compounds of general formula (IIIa) or (IIIc), wherein:
X=PLA, n+k=12 and p=13; or
X=PLAPCL, n+k=10 and p=13; or
X=PLAPCL, n+k=9 and p=13; or
X=PLAPCL, n+k=8 and p=13; or
X=PCL; n+k=8 and p=13; or
X=PLGA; n+k=12 and p=13; or
X=PCL, n+k=10 and p=4; or
X=PCL, n+k=12 and p=2.

3. The composition of claim 1, wherein the degradable block copolymer cross-linker c) is of general formula (IIIa) or (IIIc), wherein X represents PCL or PLAPCL.

4. The composition of claim 1, wherein the amount of degradable block copolymer cross-linker c) ranges from 5 mol % to 60 mol % relative to the total number of mole components a), b) and c).

5. The composition of claim 1, wherein the cyclic monomer b) is selected from the group consisting of 2-methylene-1,3-dioxolane, 2-methylene-1,3-dioxane, 2-methylene-1,3-dioxepane, 2-methylene-4-phenyl-1,3-dioxolane, 2-methylene-1,3,6-trioxocane and 5,6-benzo-2-methylene-1,3-dioxepane.

6. The composition of claim 1, wherein the hydrophilic monomer a) is selected from the group consisting of sec-butyl acrylate, n-butyl acrylate, t-butyl acrylate, t-butyl methacrylate, methylmethacrylate, N-dimethyl-aminoethyl(methyl)acrylate, N,N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate.

7. The composition of claim 1, wherein the crosslinked matrix of the hydrophilic degradable microsphere is further based on a chain transfer agent d).

8. The composition of claim 1, wherein the crosslinked matrix is further based on at least one ionised or ionisable monomer e) of general formula (V):
(CH₂═CR₁₂)-M-E (V),
wherein:
R₁₂ is hydrogen atom or a (C₁-C₆)alkyl group;
M is a single bond or a divalent radical having 1 to 20 carbon atoms,
E is a ionised or ionisable group.

9. The composition of claim 1, wherein the peptide antibiotic is a glycopeptide antibiotic or a polycationic peptides antibiotic.

10. The composition of claim 1, comprising between 10 and 300 mg/mL of a glycopeptide antibiotic, and between 2 and 10 mg/mL of a polycationic peptides antibiotic.

11. A method for preventing and/or treating infectious diseases, comprising administering a composition as defined in claim 1 by local delivery to a subject in need thereof.

12. A hydrophilic degradable microsphere for use for locally delivering an effective amount of a peptide antibiotic to a subject in need thereof, the hydrophilic degradable microsphere comprising a crosslinked matrix, the crosslinked matrix being based on at least:
d) from 10 mol % to 90 mol % of a hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, —(CH₂—CH₂—O)ₘ CH₃, —(CR₂R₃)ₘ—OH or —(CH₂)ₘ—NR₅R₆ with m being an integer from 1 to 30:
R₁, R₂, R₃, R₄, R₅ and R₆ are, independently of one another, hydrogen atom or a (C₁-C₆)alkyl group:
e) from 0.1 mol % to 30 mol % of a cyclic monomer of formula (II): 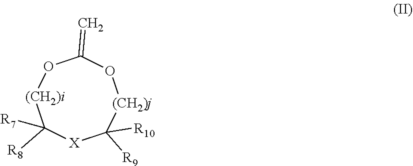
wherein:
R₇, R₈, R₉ and R₁₀ are, independently of one another, a hydrogen atom, a (C₁-C₆)alkyl group or an aryl group:
i and j are independently of one another an integer chosen between 0 and 2; and
X is a single bond or an oxygen atom:
and
f) from 5 mol % to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:
(CH₂═CR₁₁)—CO—Xₙ-PEGₚ-Xₖ—CO—(CR₁₁═CH₂) (IIIa); or
W(PEGₚ-Xₙ—O—CO—(CR₁₁═CH₂))_{z} (IIIc);
wherein
R₁₁ is independently a hydrogen atom or a (C₁-C₆)alkyl group:
Xₙ or Xₖ is independently PLA, PGA, PLGA, PCL or PLAPCL:
n and k are independently integers from 1 to 150:
W is a carbon atom, a C₁-C₆-alkyl group or an ether group comprising 1 to 6 carbon atoms:
p is an integer from 1 to 100:
z is an integer from 3 to 8:
wherein mol % of components a) to c) are expressed relative to the total number of moles of compounds a), b) and c).

13. (canceled)

14. Pharmaceutical kit comprising:
i) at least one hydrophilic degradable microsphere in association with a pharmaceutically acceptable carrier for administration by injection;
ii) an effective amount of peptides antibiotic; and
iii) optionally an injection device,
the hydrophilic degradable microsphere and the peptides antibiotic being packed separately, and wherein the hydrophilic degradable microsphere comprises a crosslinked matrix, the crosslinked matrix being based on at least:
g) from 10 mol % to 90 mol % of a hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, —(CH₂—CH₂—O)ₘ CH₃, —(CR₂R₃)ₘ—OH or —(CH₂)ₘ—NR₅R₆ with m being an integer from 1 to 30:
R₁, R₂, R₃, R₄, R₅ and R₆ are, independently of one another, hydrogen atom or a (C₁-C₆)alkyl group:
h) from 0.1 mol % to 30 mol % of a cyclic monomer of formula (II): 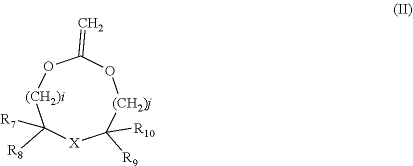
wherein:
R₇, R₈, R₉ and R₁₀ are, independently of one another, a hydrogen atom, a (C₁-C₆)alkyl group or an aryl group:
i and j are independently of one another an integer chosen between 0 and 2; and
X is a single bond or an oxygen atom:
and
i) from 5 mol % to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:
(CH₂═CR₁₁)—CO—Xₙ-PEGₚ-Xₖ—CO—(CR₁₁═CH₂) (IIIa): or
W(PEGₚ-Xₙ—O—CO—(CR₁₁═CH₂))_{z} (IIIc);
wherein
R₁₁ is independently a hydrogen atom or a (C₁-C₆)alkyl group:
Xₙ or Xₖ is independently PLA, PGA, PLGA, PCL or PLAPCL:
n and k are independently integers from 1 to 150:
W is a carbon atom, a C₁-C₆-alkyl group or an ether group comprising 1 to 6 carbon atoms:
p is an integer from 1 to 100:
z is an integer from 3 to 8:
wherein mol % of components a) to c) are expressed relative to the total number of moles of compounds a), b) and c).

15. The composition of claim 1, wherein the degradable block copolymer cross-linker c) is selected from the group consisting of compounds of general formula (IIIa), wherein:
X=PLA, n+k=12 and p=13; or
X=PLAPCL, n+k=10 and p=13; or
X=PLAPCL, n+k=9 and p=13; or
X=PLAPCL, n+k=8 and p=13; or
X=PCL; n+k=8 and p=13; or
X=PLGA; n+k=12 and p=13; or
X=PCL, n+k=10 and p=4; or
X=PCL, n+k=12 and p=2.

16. The composition of claim 1, wherein the degradable block copolymer cross-linker c) is of general formula (IIIa), wherein X represents PCL or PLAPCL.

17. The composition according to claim 8, wherein E is a ionised or ionisable group being selected from the group consisting of —COOH, —COO⁻, —SO₃H, —SO₃⁻, —PO₄H₂, —PO₄H⁻, —PO₄²⁻, —NR₁₃R₁₄, and —NR₁₅R₁₆R₁₇⁺; R₁₃, R₁₄, R₁₅, R₁₆ et R₁₇ being independently of one another hydrogen atom or a (C₁-C₆)alkyl group.

18. The composition of claim 1, wherein the peptide antibiotic is a glycopeptide antibiotic selected from the group consisting of teicoplanin, vancomycin, daptomycin, telavancin, ramoplanin, decaplanin, corbomycin, complestatin, bleomycin, oritavancin and dalbavancin.

19. The composition of claim 1, wherein the peptide antibiotic is a polycationic peptides selected from the group of polymyxins or the group of cathelicidins and defensins.
